(19) United States
(12) Patent Application Publication
(10) Pub. No.: US 2009/0042304 A1
(43) Pub. Date: Feb. 12, 2009

(54) METHOD FOR ANALYZING FOODS

(76) Inventors: Kim A. Anderson, Corvallis, OR (US); Brian W. Smith, Corvallis, OR (US)

Correspondence Address: KLARQUIST SPARKMAN, LLP, 121 SW SALMON STREET, SUITE 1600, PORTLAND, OR, 97204, US

(21) Appl. No.: 12/255,571

(22) Filed: Oct. 21, 2008

Related U.S. Application Data
(63) Continuation-in-part of application No. PCT/US2007/009682, filed on Apr. 20, 2007.
(60) Provisional application No. 60/793,909, filed on Apr. 21, 2006.

(51) Int. Cl. G01N 33/02

(52) U.S. Cl. 436/20

(57) ABSTRACT

A method for using stable isotope profiling and optionally trace element profiling to differentiate the origin of commodities, such as pistachios (Pistachia vera), or salmon, is disclosed. Isotope ratios can be determined using any suitable method, such as stable isotope mass spectrometer. Geographic regions were well separated based on isotope ratios. Seasonal effects were found to affect some isotopes for some regions.

### CROSS REFERENCE TO RELATED APPLICATION

This application is a continuation-in-part of International Application No. PCT/US2007/009682, filed on Apr. 20, 2007, which claims the benefit of the earlier filing date of U.S. Provisional Application No. 60/793,909, filed Apr. 21, 2006, both of which are incorporated herein by reference in their entirety.

### FIELD

The present disclosure concerns a method for determining information about foods, such as geographic origin, growing season, seasonal variability, climatic conditions and/or production method, using stable isotope profiling and/or chemical composition analysis.

### BACKGROUND

### I. Geographic Origin of Food Products

Geographic indications increasingly serve as a marketing tool that adds economic value to agricultural products. For example, geographic indicia convey a cultural identity by identifying a region of origin. Recognizing the value of specific human skills and natural resources in the productive process creates a unique identity for food products. The World Trade Organization (WTO) Agreement on Trade-Related Aspects of Intellectual Property Rights (TRIPS) was established to protect names of particular food products associated with certain geographic locations (Food Geographic Indications). Increasing demands on the agrifood industry from free trade, globalization, and changing technology only furthers the drive to determine food authenticity. However, financial incentives motivate dishonest retailers and re-sellers to misidentify geographic origin of commodities and food products.

### II. Production Methods of Food Products

Consumers are ever more concerned with the methods used to produce consumable food products. This is exemplified by the rise in sales of food products labeled “free range,” “organic,” or “wild caught.” Consumers need information on production methods in order to make informed decisions about the food products they choose. For example, concerns over environmental sustainability, animal welfare, health, and quality control standards in some countries may influence the buying habits of consumers. In response to consumer pressure, on Apr. 4, 2005, the United State Department of Agriculture (USDA) implemented mandatory labeling of retail fish and shellfish commodities for country of origin and method of production (e.g. wild or farm-raised) (Mattingly, USDA: 2005). However, without robust and economically feasible methods for determining these features, such rules are ineffectively enforced.

### III. Particular Food Commodity Examples

A. Pistachios

Pistachio trees (Pistacia vera) are believed to have originated in Central Asia. They were brought to the Mediterranean Basin about 2000 years ago, and were introduced to California (United States) in the 1850s. Most countries produce a couple of varieties while California produces only one variety (Kernan). Over 85% of the pistachios are grown in Iran (ca. 50%), the United States (California) (ca. 25%), and Turkey (ca. 10%). The world export market is dominated by Iran (86%), with the United States ranking second at 12%.

Variation in quality, food safety (e.g. aflatoxins), import/export fees, legal implications, and financial concerns make determining country of origin for pistachios an important consideration. This is particularly true since the world pistachio export market is valued at over 600 million U.S. dollars. In 1997, the European Union (EU) banned Iranian pistachios because their shipments exceeded allowed aflatoxin levels. The ban only lasted three months; however, aggregate imports from other countries (e.g. from the United States) dropped by 40%.

An absence of specific geographic origin information may have contributed to the overall reduction of pistachio consumption in 1997. Each country's applied tariff rates, and national laws on commodities, vary dramatically. Israeli law, for example, prohibits importing Iranian goods. Nevertheless, in 1997 evidence was presented that $10 million worth of Iranian pistachios were purchased by Israeli importers. The pistachios were sold below Israeli market value, undermining worldwide prices. As a result, pistachio producers and traders are motivated to discover objective techniques, generally chemical analysis based techniques, which are useful for determining the geographic origin of pistachios.

B. Salmon

The health benefits of eating fish and in particular salmon are well documented. Salmon is an excellent source of many nutrients and vitamins, including Vitamin E and Omega-3 fatty acids. However, not all salmon are created equal. USDA compiled statistics demonstrate that the ratio of Omega-3 fatty acids to Omega-6 fatty acids is reduced in farmed salmon versus wild salmon. Furthermore, Hites et al. (Hites et al., Science 2004, 303, 266-299) demonstrated that, on average, farmed salmon contain higher concentrations of some contaminants than wild salmon. Hites et al. also found that farmed salmon from Washington State had the lowest concentrations of contaminants compared to other farmed salmon tested.

Absent confidence in food product labeling, consumers may be wary of eating more fish in view of reports such as Hites et al. Protecting market share, reputation, and consumer confidence to pay a premium for salmon is meaningful to the industry and in particular Washington state's economy. Consumers with a preference for northwest, or pacific farmed salmon, may be discouraged from buying and eating salmon if they feel they cannot trust product labels. Methods of identifying the production origins of food products will discourage unscrupulous resellers from mislabeling salmon, increasing consumer confidence. In addition to boosting consumer confidence in food labels, food safety itself can benefit from tools that identify food product origins.

### IV. Health and Safety Concerns Associated with Food Products

Public health security and bioterrorism preparedness include protecting a nation's food supply. The U.S. Department of Homeland Security, in implementing the Bioterrorism Act in 2002, has emphasized food safety as a central concern. Establishing and maintaining knowledge about food origins are important components of securing the food supply. Authenticating food specimens to specific lots (for example shipments) or geographic regions would help ensure a safe food supply. For example, the incidence of bovine spongiform encephalitis (BSE) has led to bans on beef imports from certain regions of the world. Authenticating food specimens also would provide an important tool for forensic investigations or detention of foods that may pose a public health risk. For example, the suspected carcinogen malachite green, banned in the U.S. since 1991, has been discovered in farmed salmon imported into the U.S.

### V. Chemical and Stable Isotope Analysis

Mineral, trace element, and isotopic compositions of fruits and vegetables provide a distorted reflection of the trace mineral compositions of the soil and environment in which the plant grows. The soil-plant system is highly specific for different elements, plant species, and environmental conditions. Under most conditions, a trace element present in the vegetable/fruit must have existed in the rooting zone of the plant, at least in a slightly soluble form. Trace elements also must pass through at least one cellular membrane to move from soil to plant. The selectivity of mineral bioaccumulation processes within food products varies with different trace elements, with different plants, and with the unique environment in which the commodity is grown.

Isotopic and/or trace element profiles of animals similarly are affected by the isotopic and trace element profiles contained within the food they ingest. Factors that can affect the bioaccumulation of isotopes and trace elements include geographic origin and production method.

Most research literature regarding the geographic origin of commodities concerns analyzing vitamin content or other organic molecule content (such as amino acids, triglycerides, volatile aromatic compounds, etc.) present in the commodity. Some success (e.g. 60-90% correct classification) has been reported using vitamin and/or amino acid assays to determine geographic origin. However, vitamins (or other organic compounds) degrade (for example by enzymatic processes) from the time of harvest through storage to the time of analysis. Storage conditions may be especially important for some vitamin assays; for example, vitamin E is light sensitive, and changes in vitamin E content during storage have been reported. It is important, therefore, to develop a process for determining geographic origin of unknown samples that minimizes effects from storage conditions. Trace element profiles have been used to identify the origin of potatoes (Anderson et al., J. Agric. Food Chem. 1999, 47, 1568-1575), coffee (Anderson et al., J. Agric. Food Chem. 2002, 50(7), 2068-2075), and pistachios (Anderson and Smith, J. Agric. Food Chem. 2005, 53, 410-418).

Stable isotopes have been used to classify geographic origin of olive oil (Angersoa et al, J. Agric. Food Chem. 1999, 47, 1013-1017), milk/cheese (Fortunat et al., J. Anal. At. Spectrom., 2004, 19 (2), 227-234, Renou et al., Food Chem. 2004, 85, 63-66), wine (Almeida and Vasconncelos, J. Anal. At. Spectrom, 2001, 16, 607-611), whiskey (Parker et al., Food Chem. 1998, 63, 3, 423-428), flavors (Hor et al., J. Agric. Food Chem. 2001, 49, 21-25, Lamprecht et al., J. Agric. Food Chem. 1994, 42, 1722-1727), wheat (Branch et al., J. Anal. At. Spectrom., 2003, 18, 17-22), and orange juice (Antolovich et al., J. Agric. Food Chem. 2001, 49, 2623-2626). Various degrees of success have been reported. Many authenticity studies had an insufficient sample size to provide meaningful data or predictive abilities (less than 30 samples), and therefore conclusions concerning the effectiveness of these techniques should be made prudently. Day et al. (Day et al., J. Csi. Food Argric. 1995, 67, 113-123) combined 2D-NMR analyses with multiple elemental and isotopic ratio determinations to determine the geographic origin of wines. Although this technique correctly classified the geographic origin of wine with better than 99% accuracy, the approach requires using several instruments, including SNIF-nuclear magnetic resonance, elemental analyzer-isotope ratio mass spectrometry, flame atomic absorption spectrometry, electrothermal atomic absorption spectrometry, and inductively coupled plasma atomic emission spectrometry (ICPAES). In addition to the multiple analyses and instruments required for Day's process, sophisticated techniques were necessarily employed to determine the five isotopic ratios used.

A published report has shown that it may be possible to predict the origin of food products, such as pistachios (Anderson and Smith, J. Agric. Food Chem. 2005, 53, 410-418), using stable isotope profiling. However, this study lacked the use of databases and classification algorithms useful for widespread implementation of isotope profiling as a method for determining the origin of diverse classes of food products.

In summary, most publications concerning geographic classification have focused on complicated chemical analyses of processed foods, particularly wines and juices, and, to a lesser extent, on cocoa and olive oil. Less complicated analytical chemical methods are needed to obtain desired information from food commodities, such as to confirm food label statements concerning geographic identification and production method.

### SUMMARY

Embodiments of a method for analyzing food products are described. Certain embodiments of the method include determining stable isotope amounts, including isotope ratios of at least two isotopes of a food product, optionally determining concentration of at least one trace element of a food product, and using the isotopic and optional concentration data obtained to determine desired information concerning the food product. In some embodiments, the method includes determining the concentration of at least one trace element of a food product, and using the concentration data obtained to determine desired information concerning the food product. Examples of food products include, without limitation, plant matter such as fresh fruits, vegetables, nuts, grains, and cereals or animal matter such as fish, beef, pork, fowl, and the like. Aspects of the disclosed method are exemplified by reference to working embodiments concerning pistachios. In certain embodiments, the food product is a commodity. “Commodity” as used herein refers to a food product that has not been processed into other products or product forms, but may have been subjected to typical picking and packing processes, including washing and packaging.

With reference to food products obtained from plants, examples of information that can be obtained using the disclosed method include, but are not limited to, geographic origin, growth season, environmental conditions, seasonal variability, or combinations thereof. Seasonal variability, for example, can be determined by comparing element distributions by season for a given region.

Examples of the information that can be obtained from food products derived from animals include geographic origin and production method. With reference to fish, examples of production methods include whether the food product was obtained from a wild caught or farm raised animal from a geographically identifiable location. For example, salmon farmed on the west coast of the United States can have identifiably different isotopic and trace element profiles from salmon farmed on the east coast, both of which can have a different isotopic and trace element profiles from wild salmon. These differences originate from the different environmental conditions under which the animals live or age. For example, farmed salmon fed a specific feed obtained from one feed producer can exhibit different isotopic and trace element profiles from fish fed with feed produced from a second producer. Other examples of desired information include determining if the animal derived food products are from a free range or caged bred source.

Certain embodiments of the method also are disclosed for correlating the isotopic and/or elemental profiles of a food product to the origin of the food product. By way of example and without limitation these techniques include principal component analysis (PCA), canonical discriminant analysis (CDA), linear discriminant function analysis, quadratic discriminant function analysis, neural network modeling, genetic neural network modeling, classification trees, or combinations thereof. It is also contemplated that these correlations or “correlation data” can be stored on computer readable media for later use, such as in the form of a searchable database.

Another aspect of this disclosure concerns developing algorithms for determining food product origin. The algorithms can be constructed from correlation data. A person of ordinary skill in the art will appreciate that these algorithms can be stored on computer readable media, which can be used to implement embodiments of the disclosed method.

The foregoing and other objects, features, and advantages of the invention will become more apparent from the following detailed description, which proceeds with reference to the accompanying figures.

### BRIEF DESCRIPTION OF THE DRAWINGS

FIGS. 1A and 1B show three-dimensional plots of elemental profiles of regional origins of pistachios.

FIG. 1A shows the concentration of strontium, potassium, and magnesium versus geographic growing origin. All varieties and two growing seasons are shown (n=371).

FIG. 1B shows the concentration of strontium, copper, and iron for the 2001 season. Subregions and varieties are shown.

FIGS. 2A and 2B shows box plots of elements in pistachios.

FIG. 2A shows box plots from different growing regions. The regions are indicated in the figure.

FIG. 2B shows box plots of pistachio elements from different growing regions in the 2000 and 2001 seasons.

FIG. 3 shows score plots of the first three PCs for trace elements in pistachios from different pistachios and different growing regions.

FIG. 4 shows score plots of the first two canonical variables used to discriminate trace elements in pistachios from different growing regions.

FIG. 5 provides score plots of the first two canonical variables used to discriminate trace elements in pistachios from different growing regions and different years.

FIGS. 6A and 6B show plots of geographic origin of pistachios using the carbon and nitrogen ratios for pistachios in 2001.

FIG. 6A is a plot of location using bulk C/N ratios and δ¹⁵N‰. A subset of the Iranian samples (n=23) from four different locations, a subset of Turkish samples (n=23) from four different locations, and a subset of Californian samples (a=25) for the 2001 season are shown.

FIG. 6B is a plot of geographic location using the δ¹⁵N‰ and δ¹³C‰ for pistachios in 2001. The subsets of the three geographic regions (n=71) and the samples analyzed are the same as in FIG. 6A.

FIGS. 7A-7C show information concerning three geographic growing regions, several pistachio varieties within each region, and two growing seasons (n=146).

FIG. 7A illustrates stable isotope (δ¹⁵N‰) and bulk C/N ratio versus three geographic growing origins.

FIG. 7B illustrates a tree-based model results in a simplified hierarchical tree of decision rules useful for classification of pistachios. Use of the decision rules from the tree model results in <5% misclassification error rate. Legend is 1=USA, 2=Iran and 3=Turkey.

FIG. 7C is a plot of the first two PCs for pistachios from three different regions. Legend is 1=USA, 2=Iran and 3=Turkey.

FIGS. 8A-8C shows box plots for seasonal variation of bulk C/N ratio (FIG. 8A), δ¹⁵N‰ (FIG. 8B) and δ¹³C ‰ (FIG. 8C) from Iran and USA. The boundary of the box indicates the 25th and 75th (top and bottom) percentile. The line within the box marks the median. The whiskers above and below the box indicate the 90th and 10th percentile. All box plot outliers are displayed with the symbol.

FIGS. 9A and 9B show plots of location versus δ¹⁵N‰ and δ¹³C‰. FIG. 9A shows sub-regional geographic locations from Iran: North (▪), Central (▴), and South () and sub-location geographic designations (see legend).

FIG. 9B shows Turkish pistachios, sub-regional and sub-location geographic designations (see legend).

FIG. 10 is a plot showing variety differences in Turkish and Iranian pistachios using bulk C/N ratio versus δ¹⁵N‰.

FIG. 11 is a block diagram of a computer system that can be used to implement aspects of the present disclosure.

FIG. 12 is a diagram of a distributed computing environment in which aspects of the present disclosure can be implemented.

FIGS. 13A-13C show box plots of the element concentrations of Oregon and Mexican strawberries (FIG. 13A), Oregon and Chilean blueberries (FIG. 13B), and Oregon and Argentine pears (FIG. 13C). (A: Oregon: n=40, Mexico: n=42; Iron Oregon: n=20, Iron Mexico: n=21); (B: Oregon: n=32, Chile: n=37; Iron Oregon: n=16, Iron Chile: n=37); (C: Oregon: n=40, Argentina: n=40). Significant separation was determined using a two sample t-test. The boundary of the box indicates the 25th and 75th (top and bottom) percentile. The lines within the box mark the mean and the median. The whiskers above and below the box indicate the 90th and 10th percentile. The 5^{th} and 95^{th} percentiles are displayed with the symbol.

FIGS. 14A and 14B show plots of concentrations of copper (Cu) and manganese (Mn) in Oregon and Mexican strawberries (mg/kg) (FIG. 14A); concentrations of calcium (Ca) and manganese (Mn) in Oregon and Chilean blueberries (mg/kg) (FIG. 14B).

FIGS. 15A-15C shows plots of principal component one versus principal component 2, for chemical profile of elements in Oregon and Mexican strawberries (n=80) (FIG. 15A), Oregon and Chilean blueberries (n=68) (FIG. 15B), and Oregon and Argentine pears (n=80) (FIG. 15C).

FIGS. 16A-16C show plots of the CDA frequency chart using the first canonical variable. All 10 available dimensions are utilized in this simplified visual representation of the separation between Oregon and Mexican strawberries (n=80) (FIG. 16A), Oregon and Chilean blueberries (n=68) (FIG. 16B), and Oregon and Argentine pears (n=80) (FIG. 16C).

FIGS. 17A-17C are bar graphs showing the relative importance of inputs for Genetic Neural Network modeling used to classify Oregon and Mexican strawberries (FIG. 17A), Oregon and Chilean blueberries (FIG. 17B), and Oregon and Argentina pears (FIG. 17C).

FIG. 18 shows the hierarchal tree models for classification of Oregon and Mexican strawberry samples, Oregon and Chilean blueberry samples, and Oregon and Argentine pear samples. A tree-based model results in a simplified hierarchical tree of decision rules useful for classification of pears. Use of the decision re-substitution rules results in a 100%, 100%, and 93% correct classification rate respectively for this data set.

FIG. 19 shows a plot of Argentina pear and Oregon pear isotope ratios organized by region (Oregon n=16, Argentina n=20).

FIGS. 20A-20C show plots of strawberry (FIG. 20A), blueberry (FIG. 20B), and pear (FIG. 20C) copper and manganese concentrations organized by subregion and variety. Statistical differences were determined using a multiple comparisons ANOVA. Letters denote statistical differences, 0.95 confidence level. The boundary of the box indicates the 25th and 75th (top and bottom) percentile. The solid lines in the box mark the median and mean. The 5^{th} and 95^{th} percentile are displayed with the symbol.

FIG. 21 shows a plot of the first two canonical variables from the correlation of isotope ratios to the origin of “wild” and farm raised salmon.

FIG. 22 shows a plot of the first two canonical variables from the correlation of trace metal concentration to the origin of “wild” and farm raised salmon.

### DETAILED DESCRIPTION

### I. Method of Determining the Origin of Food Products

The present disclosure concerns embodiments of a method for determining the origin of food products. Certain disclosed embodiments include determining the stable isotope ratios of food products and/or determining the concentration of trace elements in a food product. Certain embodiments also include correlating isotope ratios and elemental concentrations to the origin of a food product, and predicting the origin of a food product of previously undetermined origin.

### II. Correlation of Isotope Ratios to Food Product Origin

Aspects of the method disclosed herein concern correlating stable isotope ratios of a food product to the origin of the food product. Typically, a food product of known origin is provided and stable isotope ratios are determined for the food product that can be correlated with the origin of the food product. Specific techniques are provided herein for determining stable isotope ratios of the food product. However, it should be noted that stable isotope ratios can be determined by any suitable technique, including but not limited to, mass spectrometry.

A person of ordinary skill in the art will appreciate that any isotope having a sufficient concentration in a food product to be detectable potentially can be used to practice the disclosed method. Thus, by way of example, and without limitation, stable isotopes that are detectable and may be used to deduce characteristics of food products include ¹³C, ¹²C, ¹⁵N, ¹⁴N, ¹⁸O, ¹⁶O, ²H, ¹H. It also may be advantageous to detect isotopes of elements other than those listed above.

In several non-limiting examples, the data obtained for the food product includes determining at least one stable isotope ratio of the food product. A person of ordinary skill in the art would recognize that at least one isotope ratio includes any and all isotope ratio integers greater than zero, for example 1, 2, 3, etc. In certain embodiments of the disclosed method, absolute amounts of such isotopes can be determined.

¹³C, ¹²C, ¹⁵N, ¹⁴N are common isotopes that are used to practice the disclosed method. For example, working embodiments have determined δ¹³C by measuring CO₂ and have determined δ¹⁵N by measuring N₂.

In a specific disclosed example, δ¹⁵N‰ values for geographic regions were statistically different for pistachios. Working embodiments had δ¹⁵N‰ values ranging from about −3 to about 10. For example: Turkish δ¹⁵N‰ pistachio values typically range from about −2 to about +3.0; USA δ¹⁵N‰ pistachio values typically range from about 0 to about +2.5; and Iranian δ¹⁵N‰ pistachio values typically range from about +1 to about +9.

Bulk C/N ratios can also be correlated to food product origin. For example in pistachios, bulk C/N values ranged from about 13 to about 23: bulk C/N ratios for Turkish pistachios typically range from about 18 to about 23; USA bulk C/N ratios for pistachios typically range from about 6 to about 16; and Iranian bulk C/N ratios for pistachios typically range from about 16 to about 23. In still other embodiments, bulk C/N ratios versus δ¹⁵N are used to determine geographic origin.

Isotopes selected for ratio determination in a food product may depend on factors such as, but not limited to, geographic origin, crop type, crop variety, season, and feed. As disclosed herein, isotope ratios of the food product can be correlated to the origin of the food product, where the term “origin” or “food product origin” includes but is not limited to geographic origin, climatic origin, seasonal origin, environmental origin, and combinations thereof. In certain embodiments, “origin” may reflect production method. Examples of production methods include, with out limitation, farmed, wild, free range, and caged. Thus, it is understood that isotope ratios may be correlated to geographic origin, climatic origin, seasonal origin, environmental origin, production method, and combinations thereof.

It is further understood that the correlation between isotope ratios and food product origin can be used to predict the origin of food product where the origin of growth or production is unknown.

### III. Correlation of Trace Elements to Food Product Origin

In addition to isotope profiling, aspects of the disclosed method concern correlating trace element concentrations to food product origin. Techniques are provided herein for determining trace element concentrations in a food product and for correlating trace element concentrations to the origin of a food product.

Any element that is accumulated by the plant or animal and is present in the food product in sufficient amount to be detectable can be used to practice the disclosed embodiments. Trace elements that can be correlated to food product origin include, without limitation, Ca, Cu, Fe, K, Mg, Mn, Na, P, Sr, V, Zn, and combinations thereof.

A person of ordinary skill in the art will understand that the concentration of elements other than those listed also may be determined. Further, the measured profile of trace element concentrations found in a food product may depend on factors such as, but not limited to, geographic origin, crop type, crop variety, season, and production method.

In certain embodiments, the trace element concentrations are correlated to food product origin using statistical models. Examples of statistical models include principal component analysis (PCA), canonical discriminant analysis (CDA), linear discriminant function analysis, neural network modeling, genetic neural network modeling, and hierarchical trees. Combinations of these statistical techniques also can be used.

Certain working examples of the disclosed method have used PCA as applied to Sr, Fe, Cu, K, Na, Mg, Mn, or P. The first principal component (PC) accounts for the majority of total variation and includes concentrations of Sr, Fe, and Cu. The second PC includes concentrations of K, Na, and Cu. The third PC includes Mg, Mn, or P. It will, however, be appreciated by one of ordinary skill in the art that elements selected for inclusion in the first, second, third, etc. principle component may depend upon such factors as the food product undergoing trace element determination. PCA has also been applied to normalize trace element data.

In some embodiments, canonical discriminant analysis (CDA) is used to obtain group clustering. For example, for certain working embodiments of the method concerning pistachios, CDA was applied to Sr, Cu, Na, Ca, Fe, and Cu concentrations. The elements having the largest effect on the first canonical variable include Sr, Cu, and Na. Those elements having the largest effect on the second canonical variable include Ca, Fe, and Cu.

Determining concentration of at least one element of the food product is optional. Disclosed embodiments also can include determining both (1) stable isotope ratios of at least two isotopes, and (2) trace element concentration of at least one trace element (preferably concentrations of plural trace elements).

Certain embodiments are directed to determining concentrations of plant macroelement concentrations and/or ratios of concentrations, which can vary from plant-to-plant. Macroelements typically include calcium, potassium, magnesium, phosphorous, or combinations thereof.

Other embodiments involve analyzing combinations of trace elements, such as: potassium, magnesium, and strontium; or copper, iron, manganese, vanadium, and zinc. Again with reference to pistachios and determining, for example geographic origin, copper amounts ranged from as low as about 5 μg/g to at least about 13 μg/g; iron ranges were from at least as low as 20 μg/g to at least about 50 μg/g; manganese ranges were from at least as low as about 9 μg/g to at least about 15 μg/g; vanadium ranges were from at least as low as about 4 μg/g to at least about 21 μg/g; and zinc concentration ranges were from at least as low as about 17 μg/g to at least about 37 μg/g.

### IV. Determination of Food Product Origin

Aspects of the current disclosure concern embodiments of a method for determining the origin of a food product of unknown origin. Typical embodiments proceed by determining the stable isotope ratio or profile of a food product of unknown origin, and/or determining the trace element concentrations or profile of the food product of unknown origin. The isotopic and/or trace element profile of the food product of unknown origin is then compared to the isotopic and/or trace element profile of a food product of known origin. The origin of the food product of unknown origin then can be determined by such comparison.

It will be appreciated by one of ordinary skill in the art that any method that accurately predicts the origin of the food product of unknown origin may be employed. Such methods may include but are not limited to visual inspection of the data, the use of a categorization tree/algorithm to classify origin, suitable analytical processes, including principal component analysis (PCA), canonical discriminant analysis (CDA), linear discriminant function analysis, quadratic discriminant function analysis, neural network modeling, genetic neural network modeling, categorization trees, or other computational energy minimization methods such as simulated annealing, Powel minimization, conjugate direction minimization, maximum likelihood, or steepest dissent minimization, and any or all combinations thereof. In certain embodiments, the food product of known origin will include a representative sample of the food product of known origin, such that statistical parameters describing the representative sample can be calculated. The calculation of statistical parameters such as mean, standard deviation, variance, and the like is well known in the art.

The data obtained practicing the disclosed method can be analyzed by a variety of suitable methods, such as statistical methods, that facilitate analyzing and/or conveying the desired information. Certain embodiments include determining the concentration of at least one trace element and applying canonical discriminant analysis to obtain group clustering. Three-dimensional plots of data, such as trace element composition data, trace element concentration data, concentration ratio data, isotope composition data, isotope concentrations data, or combinations thereof, also can be used to determine and/or convey the desired information. For example, working embodiments have used a three-dimensional plot of strontium, iron, and copper concentrations to determine food product origin.

One particular disclosed example of the method concerns analyzing commodities. The method comprises providing a food commodity and optionally, but most typically, determining concentrations of plural trace elements of the food commodity, including at least strontium concentrations. Stable isotope ratios of two or more stable isotopes of the commodity, including at least ¹³C and ¹⁵N, are determined, such as by mass spectrometry. Desired information, such as geographic origin, growth season, environmental conditions, or combinations thereof, is then determined from the element concentration and/or isotope data using suitable analytical processes, including principal component analysis (PCA), canonical discriminant analysis (CDA), linear discriminant function analysis, quadratic discriminant function analysis, neural network modeling, genetic neural network modeling, or combinations thereof.

### V. Algorithms

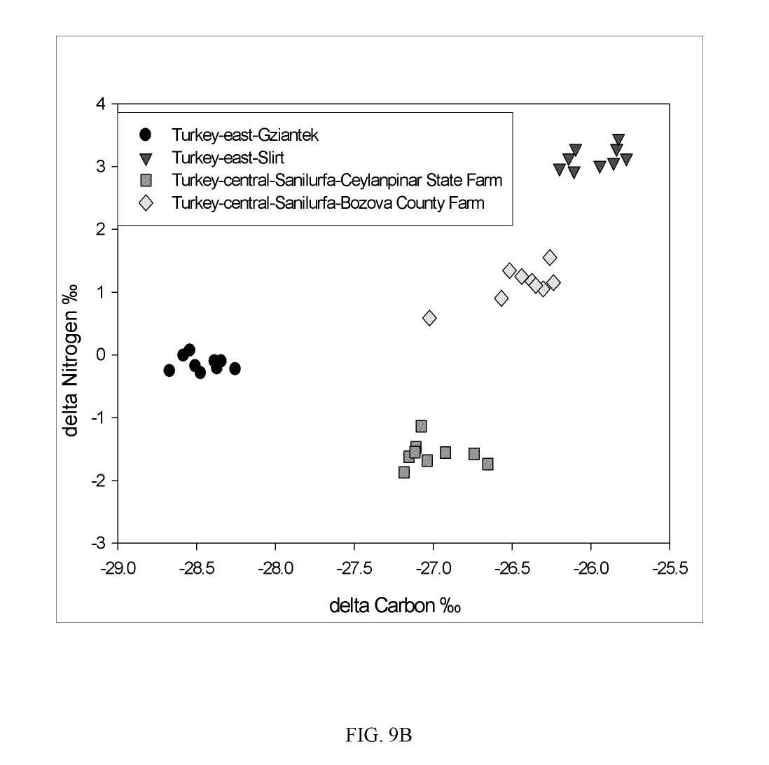

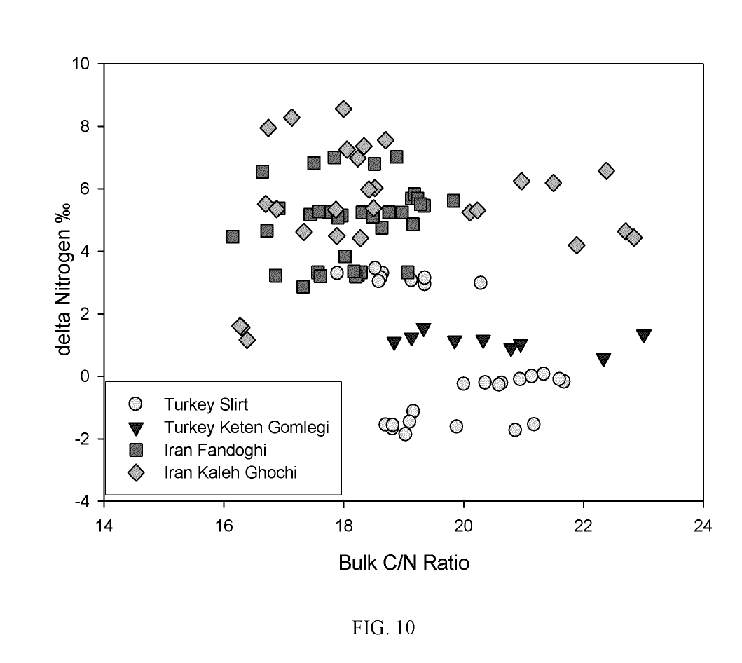

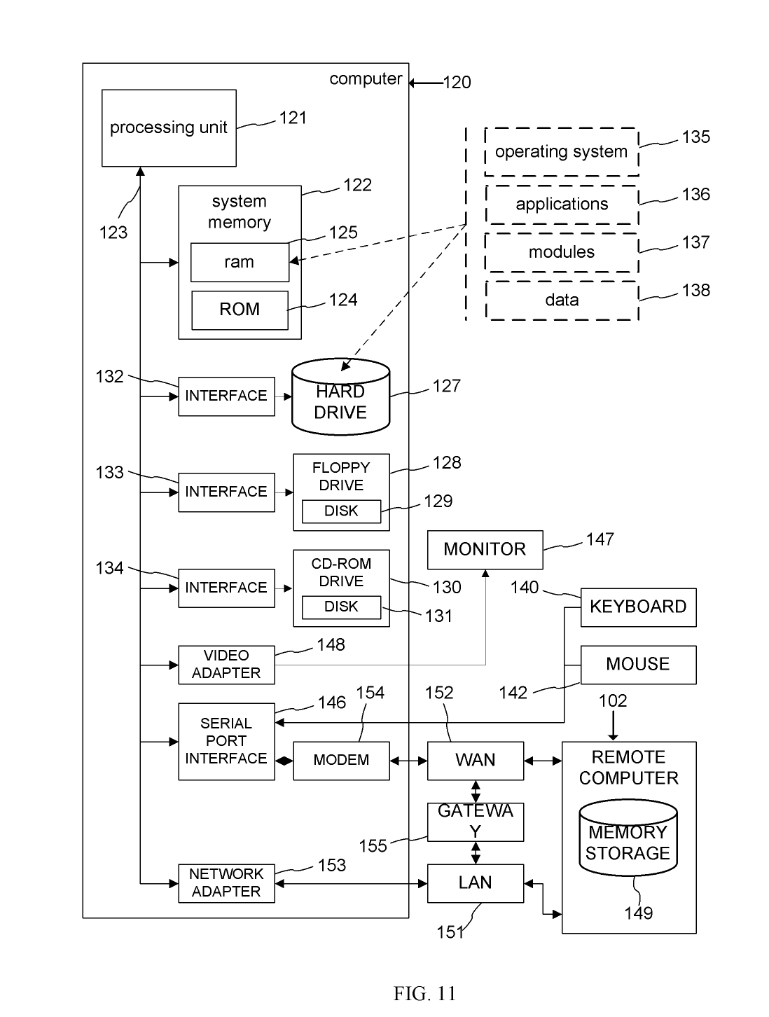

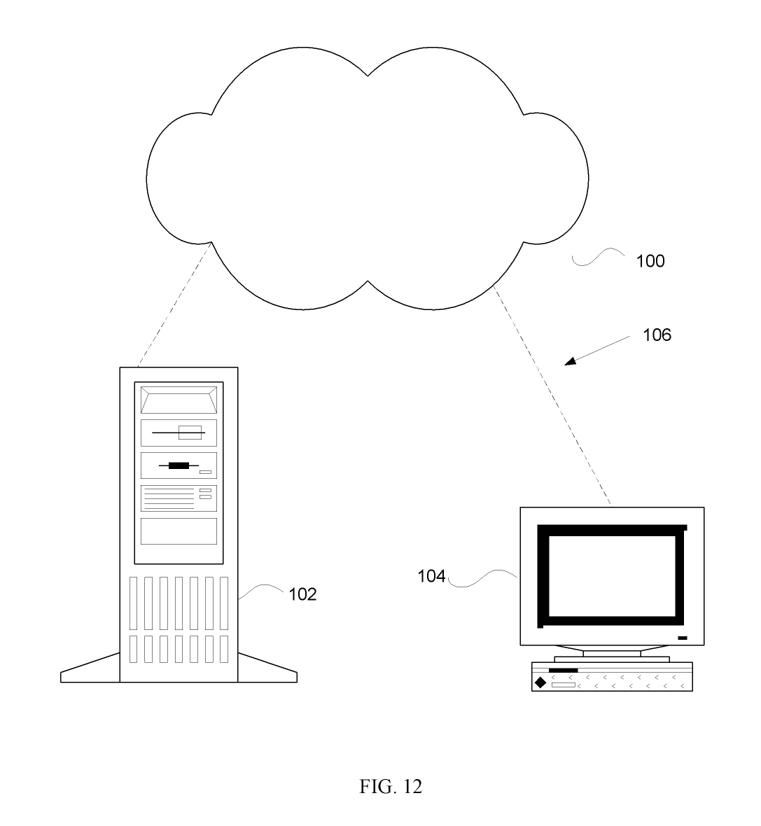

Aspects of the disclosed method concern the construction of algorithms to predict the origin of a food product. Accordingly, a method is disclosed herein for constructing a categorization tree/algorithm for predicting the origin of a food product of unknown origin. In a particular disclosed example, a categorization tree/algorithm for determining pistachio origin was constructed. This algorithm included two variables, three decision nodes, and is capable of classifying pistachios from the USA, Iran, and Turkey with greater than 95% accuracy (see FIG. 7B). In other disclosed examples, trace element concentrations were used to construct algorithms for determining the origins of pears, blueberries, and strawberries.

Typically, an algorithm is constructed by providing a data set wherein the origin(s) of a food product has been correlated with the isotopic and trace elemental profile of the food product. A rule set is determined to enable one of ordinary skill in the art to determine the origin of a food product of previously unknown origin with a high degree of certainty. By providing a food product of unknown origin, determining the isotopic profile and/or the trace element profile of the food product of unknown origin, algorithms of this disclose can be used to predict the origin of a food product of unknown origin.

In a particular example, not bounded by theory, the classification tree or algorithm is fitted using binary recursive partitioning in which the data are successively split along coordinate axes of the predictor variables, such that at any node, the split which maximally distinguishes the response variable in the left and the right branches is selected. Splitting continues until nodes are pure or data are too sparse; terminal nodes are called leaves, while the initial node is called the root. In this example, the model used for classification assumes that the response variable follows a multinomial distribution, and that the data is not weighted in the computation of the deviance.

Algorithms can be stored on a computer readable media for immediate or later use. Such algorithms also can be translated into a set of instructions readable and capable of being executed by a computer, such that the isotopic and trace element profiles of a food product of unknown origin can be entered into the computer and the origin predicted from these values using the classification trees/algorithms. The algorithms can be integrated into a hand held device for determining the isotopic ratios and/or trace element profiles of a food product.

### VI. Databases

Aspects of the method disclosed herein concern databases of isotopic and trace elements profiles correlated to food product origin. Data correlating food product origin to the isotopic and trace element profile can be stored in a machine-readable format for later use, such as in a database. The present disclosure also provides for a machine-readable data storage medium, which comprises a data storage material encoded with machine readable data defining the correlation of food product origin to the isotopic ratios and trace element profile. Machine readable data storage material can be used to predict the origin of a food product using a computer, computer program, or other method.

A database can be generated by providing at least one food product of known origin, determining at least one stable isotope ratio, and/or determining the concentration of at least one trace element the food product thereby creating isotopic and trace element profiles of the food product. The isotopic and trace element profiles can be correlated to the origin of the food and assembled into a database.

In some circumstances, trace element profiles or isotopic profiles will be unavailable for a food product. One of ordinary skill in the art will appreciate that under these circumstances a database will have a null entry for these values, indicative of no data available for that entry.

A. Database Construction and Consultation for Origin Identification

In its various embodiments, the method presented herein can be utilized for both assembling a database of isotopic and/or trace element profiles correlated to the origin of a food products of known origin and consulting such databases for identifying the origin of a food product of unknown origin. Assembly/generation and consultation of such databases may be automated using a computer executable software program.

1. Database Assembly

The databases of the present disclosure allow the rapid identification of the origin of a food product of unknown origin by making it possible to identify the origin of the food product based upon the isotopic profiles and trace element profiles. For example, the origin of a sample of pistachios can be predicted based on the isotopic profile and/or the trace element profile by comparison with the isotope and trace element profiles of pistachios of know origin maintained in a database.

A database as is a dynamic data structure, and isotopic and/or trace element profiles can be added to the database as need be. For example an isotope and trace element profile for a food product from a new origin can be added to the database or the isotope and trace element profile of a food product previously not represented in the database may be added.

Databases can be accessed though a user interface. Examples of such a user interface include, without limitation, electronic devices, such as a computer or a hand held device. The databases of the present disclosure can be stored locally, such on the computer or hand held device, or remotely, such as on a file server or main frame computer. It is also an aspect of this disclosure that a fee for access to the database can be charged.

2. Database Consultation for Identifying Food Product Origin

The isotopic and trace element profile of the food product of unknown origin can be compared to the isotopic and trace element profile database to identify the origin of the food product of unknown origin. This can be done using RESolve, STATISTICA™, Pirouette, SAS® Version 8, S-PLUS® or any other pattern recognition program, including an artificial neural network, genetic neural network modeling, for example programs from Ward Systems Group Inc. Typically, the program makes a comparison between the isotopic and trace element profile exhibited by the food product of unknown origin and the isotopic and trace element profiles exhibited by food products of known origin stored in the database. The origin of the food product of unknown origin can be predicted to be the same as the origin as the food product with the most similar isotopic and trace element profile. Similarity may be judged, for example, by proximity of the isotopic and trace element profile on a CV score plot if the number of possible identities has been reduced to 4 or 5 nearest neighbors. Alternatively, similarity may be judged by algebraic and statistical methods well known in the art and embodied as standard features in available software pattern recognition packages as predictions of the likelihood of origin. In one embodiment, the isotope and trace element profile of a food product of know origin is measured, and can be analyzed using a pattern recognition program to generate principal components and canonical variables representing the data. Following such analysis, each isotope and trace element profiles a food product of know origin can be represented as a point in multi-dimensional space, where the principal components or canonical variables are the axes of that space.

By way of illustration, if two food products of different origins have different isotope and trace element profiles they will be represented by two points separated in multidimensional space. A vector defined, for example, as connecting the point representing isotope and trace element profiles from one origin to the point representing its isotope and trace element profiles from a second origin can be determined for each food product. Similarities between the directions and the lengths of these vectors may be detected by pattern recognition and the origins grouped according to the similarities of their vectors.

3. Exemplary Computer System

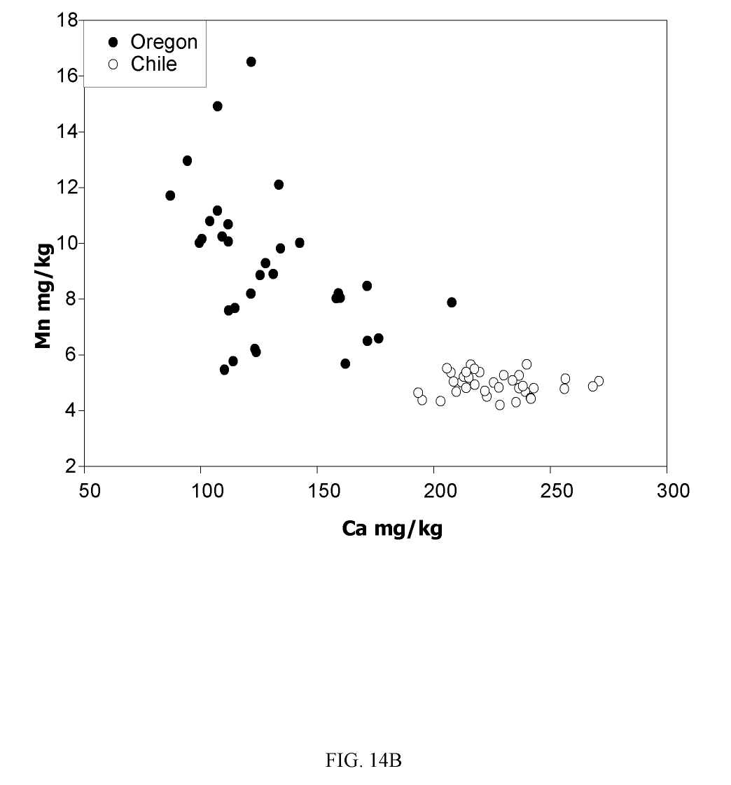

FIG. 11 illustrates an exemplary computer system 120 that can serve as an operating environment for the software for determining food product origin and database storing the isotopic and trace element correlation data. With reference to FIG. 11 an exemplary computer system for implementing the disclosed method includes a computer 120 (such as a personal computer, laptop, palmtop, set-top, server, mainframe, hand held device, and other varieties of computer), including a processing unit 121, a system memory 122, and a system bus 123 that couples various system components including the system memory to the processing unit 121. The processing unit can be any of various commercially available processors, including INTEL® x86, PENTIUM® and compatible microprocessors from INTEL® and others, including Cyrix, AMD and Nexgen; Alpha from Digital; MIPS from MIPS Technology, NEC, IDT®, Siemens, and others; and the PowerPC from IBM® and Motorola. Dual microprocessors and other multi-processor architectures also can be used as the processing unit 121.

The system bus can be any of several types of bus structure including a memory bus or memory controller, a peripheral bus, and a local bus using any of a variety of conventional bus architectures such as PCI, VESA, AGP, Microchannel, ISA and EISA, to name a few. A basic input/output system (BIOS), containing the basic routines that help to transfer information between elements within the computer 120, such as during start-up, is stored in ROM 124. The system memory includes read only memory (ROM) 124 and random access memory (RAM) 125.

The computer 120 may further include a hard disk drive 127, a magnetic disk drive 128, for example to read from or write to a removable disk 129, and an optical disk drive 130, for example to read a CD-ROM disk 131 or to read from or write to other optical media. The hard disk drive 127, magnetic disk drive 128, and optical disk drive 130 are connected to the system bus 123 by a hard disk drive interface 132, a magnetic disk drive interface 133, and an optical drive interface 134, respectively. The drives and their associated computer readable media provide nonvolatile storage of data, data structures (databases), computer executable instructions, etc. for the computer 120. Although the description of computer readable media above refers to a hard disk, a removable magnetic disk and a CD, it should be appreciated by those skilled in the art that other types of media which are readable by a computer, such as magnetic cassettes, flash memory cards, digital video disks, Bernoulli cartridges, and the like, can also be used in the exemplary operating environment.

A number of the isotope and trace element profiles can be stored in the drives and RAM 125, including an operating system 135, one or more application programs 136, other program modules 137, and program data 138.

A user can enter commands and information into the computer 120 using various input devises, such as a keyboard 140 and pointing device, such as a mouse 142. Other input devices (not shown) can include a microphone, satellite dish, scanner, or the like. These and other input devices are often connected to the processing unit 121 through a serial port interface 146 that is coupled to the system bus, but can be connected by other interfaces, such as a parallel port, game port or a universal serial bus (USB). A monitor 147 or other type of display device is also connected to the system bus 123 via an interface, such as a video adapter 148. In addition to the monitor, computers typically include other peripheral output devices (not shown), such as printers.

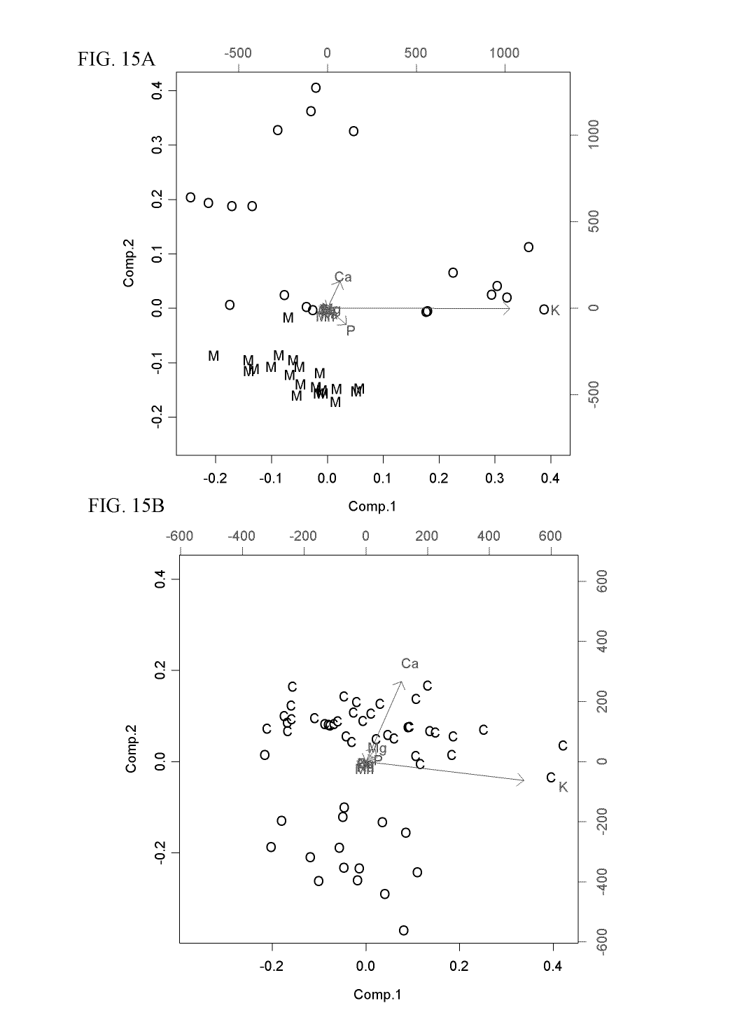

The computer 120 can operate in a networked environment using logical connections to one or more other computer systems, such as computer 102. The other computer systems can be servers, routers, peer devices or other common network nodes, and typically include many or all of the elements described relative to the computer 120, although only a memory storage device 149 has been illustrated in FIG. 12. The logical connections depicted in FIG. 12 include a local area network (LAN) 151 and a wide area network (WAN) 152. Such networking environments are common in offices, enterprise-wide computer networks, intranets and the Internet.

When used in a LAN networking environment, the computer 120 is connected to the local network 151 through a network interface or adapter 153. When used in a WAN networking environment, the computer 120 typically includes a modem 154 or other means for establishing communications (for example via the LAN 151 and a gateway or proxy server 155) over the wide area network 152, such as the Internet. The modem 154, which can be internal or external, is connected to the system bus 123 via the serial port interface 146. In a networked environment, program modules depicted relative to the computer 120, or portions thereof, can be stored in the remote memory storage device. It will be appreciated that the network connections shown are exemplary and other means of establishing a communications link between the computer systems (including an Ethernet card, ISDN terminal adapter, ADSL modem, 10BaseT adapter, 100BaseT adapter, ATM adapter, or the like) can be used.

The methods, including the acts and operations they comprise, described above can be performed by the computer 120. Such acts and operations are sometimes referred to as being computer executed. It will be appreciated that the acts and symbolically represented operations include the manipulation by the processing unit 121 of electrical signals representing data bits which causes a resulting transformation or reduction of the electrical signal representation, and the maintenance of data bits at memory locations in the memory system (including the system memory 122, hard drive 127, floppy disks 129, and CD-ROM 131) to thereby reconfigure or otherwise alter the computer system's operation, as well as other processing of signals. The memory locations where data bits are maintained are physical locations that have particular electrical, magnetic, or optical properties corresponding to the data bits.

4. Exemplary Distributed Computing Environment

FIG. 12 illustrates a distributed computing environment in which the software and/or database elements used to implement the methods of the present disclosure may reside. The distributed computing environment 100 includes two computer systems 102, 104 connected by a connection medium 106, although the disclosed method is equally applicable to an arbitrary, larger number of computer systems connected by the connection medium 106. The computer systems 102, 104 can be any of several types of computer system configurations, including personal computers, multiprocessor systems, handheld devices, and the like. In terms of logical relation with other computer systems, a computer system can be a client, a server, a router, a peer device, or other common network node. Additional computer systems 102 or 104 may be connected by an arbitrary number of connection mediums 106. The connection medium 106 can comprise any local area network (LAN), wide area network (WAN), or other computer network, including but not limited to Ethernets, enterprise-wide computer networks, intranets and the Internet.

Portions of the software for determining food product origin as well as databases storing the isotopic and trace element correlation data can be implemented in a single computer system 102 or 104, with the application later distributed to other computer systems 102, 104 in the distributed computing environment 100. Portions of the software for determining food product origin may also be practiced in a distributed computing environment 100 where tasks are performed by a single computer system 102 or 104 acting as a remote processing device that is accessed through a communications network, with the distributed application later distributed to other computer systems in the distributed computing environment 100. In a networked environment, program modules comprising the software for determining food product origin as well as databases storing the isotopic and trace element correlation data can be located on more than one computer system 102 or 104. Communication between the computer systems in the distributed computing network may advantageously include encryption of the communicated data.

### VII. Chemical Trace Element Analysis for Determining Desired Information for Food Products Introduction and Data Analysis

Unidiscriminant and multidiscriminant data exploration and analysis methods were used to determine growing origin of food products, particularly fresh commodities, exemplified herein by particular reference to pistachios. Certain disclosed embodiments determined growing origin based on the measured profile of trace element concentrations found in the food samples. The methods employed include principal component analysis (PCA), canonical discriminant analysis (CDA), linear discriminant function analysis, neural network modeling, and genetic neural network modeling. Initial trace element screening included all of the elements analyzed. Elements not showing significant concentrations were not used in the subsequent data analysis. The ultimate elements included Ca, Cu, Fe, K, Mg, Mn, Na, P, Sr, V, and Zn.

Using the approach outlined above, statistics also were computed for the data grouped by growing season for a given location. Seasonal comparisons could be made within the location groups for California and Iran since these locations had two seasons of data. Data for Turkey was only available for the 2001 growing season. The unidiscriminant statistics mean, standard deviation (SD), and sample number were computed for each element by location. These data were displayed visually with box plots. Additional information included in the box plots was upper-quartile, lower quartile, standard error, maximum, and minimum.

A. Data Standardization

To ensure that different measurement scales did not affect the multidiscriminant analysis, the data were standardized by subtracting sample means and then dividing the resulting difference by the corresponding SDs.

B. Software

The software used to perform the multidiscriminant analysis was SAS® version 8e for WINDOWS®.

C. PCA

PCA generates principal components (PCs) that are linear combinations of the original variables. The first PC summarizes the maximum possible variation that can be projected onto one dimension; the second PC captures the second most and so on. The PCs are orthogonal in the original space of variables, and the number of PCs can equal the number of the original variables. However, it is sometimes the case that a large percentage of the total variation can be explained by the first few PCs, effectively reducing the number of variables needed to describe variation between individual samples. In this case, plotting the samples with respect to two or three PCs facilitates two- or three-dimensional views of how individual samples differ from one another (in the variation sense).

For a geographic classification task, it is desirable to have group differences explicitly manifest with a low-dimensional view. However, this is not always the case since this method measures variation in the elemental concentrations in the samples but does not take into account group (geographic origin) membership.

D. CDA

CDA was used to obtain the best group clustering. CDA is a dimension reduction technique related to PCA, but unlike PCA, predefined groups are included in the calculations. CDA generates canonical variables, which are linear combinations of the original variables that describe the variation between prespecified classes in a manner analogous to the way in which PCA summarizes the variation between individual samples. CDA can effectively reduce the number of variables and provide optimum low-dimensional views of the data, which display the maximum possible variation between different groups and the minimum possible variation within the same group. The number of possible canonical variables is the minimum of the number of classification groups minus one and the number of independent variables. CDA has previously been applied to data for the purpose of geographical classification of potatoes (Anderson et al., J. Agric. Food Chem. 1999, 47, 1568-1575), coffee (Anderson et al., J. Agric. Food Chem. 2002, 50(7), 2068-2075), and wine (Day et al., J. Csi. Food Argric. 1995, 67, 113-123).

E. Classification Models

Discriminant function analysis refers to a group of pattern recognition classification methods that use known data to determine a discriminant function, which can then be used to classify unknown samples into predetermined classes. Two types of discriminant functions were used: a linear discriminant function and a quadratic discriminant function. Details on how each of these methods work can be found in the description of the DISCRIM procedure in the SAS® technical manual. See, SAS® Systems for WINDOWS®, Release 6.11; SAS® Institute, Inc., Cary, N.C., (2003), incorporated herein by reference to the extent these methods are disclosed. To estimate classification accuracy, a cross-error rate was calculated. A discriminant function is constructed, leaving out one sample as an “unknown.” The sample is then classified. This process is repeated for every sample, and the cross-validation classification accuracy estimate obtained by taking the percentage of samples classified correctly.

Chemical trace element compositional analysis of foods provides a scientific foundation to geolocate commodities (such as foods) on the basis of their chemical compositions. Other geographic authenticity approaches require using several instruments. One feature of particular disclosed embodiments is that all of the elemental chemical data can be determined with the use of a single analytical instrument, an Inductively-Coupled Plasma Atomic Emission Spectrometer (ICPAES). A person of ordinary skill in the art also will appreciate that other analytical chemical methods can be used to determine trace element composition and/or concentration. However, for particular disclosed ICPAES embodiments, the data are used directly from the ICPAES into the computational models. No prior mathematical or interpretive data analyses are required, as is not often the case with other geographic authenticity approaches. In this study, IS elements were determined, unlike chromatography techniques, elemental spectroscopy data analysis requires little analyst time and only moderate expertise.

In contrast, organic acid and inorganic anion data from the capillary electrophoresis technique did not provide data that was usable for geographic profiling. Variations in organic acid and inorganic anion concentrations within each region were much larger than any differences seen between geographic regions.

1. Element Analysis

With reference to Table 1, of the 15 elements tested, 11 were routinely above detection limit.

TABLE 1
Mean Concentrations (□g/g) and SDs Dry Weight for 11 of the 15 Elements Determined in Pistachios

| | | | | | | | | |
|---|---|---|---|---|---|---|---|---|
| country/ | | | Ca avg | Cu avg | Fe avg | K avg | Mg avg | Mn avg |
| subregion | variety | n | (SD) | (SD) | (SD) | (SD) | (SD) | (SD) |

Iran 2001

| | | | | | | | | | | | | | |
|---|---|---|---|---|---|---|---|---|---|---|---|---|---|
| central | Kaleh ghochi | 20 | 1752 | (663) | 9.4 | (1.7) | 31.3 | (7.6) | 15891 | (2615) | 1984 (295) | 10.7 | (3.7) |
| central | Fandoghi | 20 | 2890 | (746) | 7.1 | (1.8) | 40.8 | (10.2) | 9164 | (1465) | 1801 (222) | 15.4 | (4.3) |
| north | Fandoghi | 20 | 1079 | (298) | 13.1 | (2.4) | 42.2 | (13.7) | 17549 | (3087) | 1476 (177) | 10.5 | (2.3) |
| north | Fandoghi | 20 | 3794 | (953) | 7.5 | (2.7) | 47.7 | (13.9) | 10174 | (821) | 1596 (161) | 14.2 | (5.1) |
| north | Fandoghi | 20 | 2313 | (1149) | 11.2 | (2.9) | 38.9 | (8.2) | 10323 | (1303) | 2053 (177) | 15.6 | (4.2) |
| north | Kaleh ghochi | 20 | 2097 | (640) | 8.5 | (1.7) | 33.0 | (6.5) | 10270 | (1780) | 1416 (139) | 9.4 | (2.5) |
| south-central | Kaleh ghochi | 20 | 1547 | (577) | 8.3 | (1.7) | 35.2 | (12.8) | 8591 | (1748) | 1384 (226) | 10.6 | (4.7) |

Iran 2000

| | | | | | | | | | | | | | |
|---|---|---|---|---|---|---|---|---|---|---|---|---|---|
| central | Fandoghi | 20 | 1367 | (526) | 8.3 | (5.2) | 24.7 | (10.7) | 9801 | (6413) | 1697 (837) | 11.3 | (5.4) |
| central | Fandoghi | 20 | 1282 | (632) | 6.0 | (1.8) | 28.8 | (12.2) | 7286 | (3936) | 1667 (455) | 11.2 | (5.4) |
| central | Kaleh ghochi | 20 | 1087 | (456) | 9.1 | (2.0) | 25.1 | (4.6) | 15212 | (5089) | 1063 (163) | 6.7 | (1.9) |
| south-central | Kaleh ghochi | 20 | 3090 | (620) | 6.9 | (2.4) | 47.6 | (6.2) | 9863 | (7332) | 1674 (169) | 17.5 | (3.8) |
| south-central | Kaleh ghochi | 20 | 2349 | (709) | 6.8 | (1.4) | 41.2 | (5.9) | 10981 | (1284) | 1527 (187) | 14.6 | (3.0) |

Turkey 20001

| | | | | | | | | | | | | | |
|---|---|---|---|---|---|---|---|---|---|---|---|---|---|
| east | Siirt | 18 | 2934 | (810) | 9.9 | (1.4) | 32.7 | (10.3) | 10623 | (1248) | 1688 (211) | 8.6 | (2.3) |
| central | Siirt | 20 | 1453 | (519) | 12.0 | (3.1) | 29.2 | (9.9) | 10339 | (1348) | 1402 (154) | 8.4 | (2.0) |
| central | Siirt | 20 | 2514 | (660) | 8.5 | (1.8) | 24.0 | (7.6) | 10340 | (1418) | 1604 (252) | 10.2 | (3.8) |
| central | Keten gomlegi | 21 | 2899 | (594) | 9.0 | (1.9) | 36.5 | (11.1) | 9020 | (1848) | 1448 (178) | 13.1 | (2.7) |

United States

| | | | | | | | | | | | | | |
|---|---|---|---|---|---|---|---|---|---|---|---|---|---|
| CA 2000 | Kernan | 20 | 1123 | (566) | 16.2 | (7.7) | 54.7 | (15.1) | 9950 | (4143) | 1567 (634) | 11.6 | (4.7) |
| CA 2001 | Kernan | 30 | 1064 | (393) | 12.0 | (4.9) | 37.0 | (10.3) | 7896 | (2033) | 1049 (256) | 11.8 | (4.1) |

| | | | | | | |
|---|---|---|---|---|---|---|
| country/ | | Na avg | P avg | Sr avg | V avg | Zn avg |
| subregion | variety | (SD) | (SD) | (SD) | (SD) | (SD) |

Iran 2001

| | | | | | | | | | | | |
|---|---|---|---|---|---|---|---|---|---|---|---|
| central | Kaleh ghochi | 137.0 | (64.6) | 8164 | (1763) | 23.4 | (8.9) | 7.2 | (1.3) | 24.2 | (6.4) |
| central | Fandoghi | 82.5 | (31.6) | 7838 | (1332) | 22.4 | (8.6) | 6.2 | (1.1) | 36.9 | (12.4) |
| north | Fandoghi | 248.3 | (89.1) | 5672 | (694) | 25.4 | (5.8) | 20.8 | (1.5) | 26.7 | (6.5) |
| north | Fandoghi | 18.5 | (6.6) | 9467 | (1865) | 49.5 | (14.5) | 4.9 | (0.7) | 36.3 | (16.4) |
| north | Fandoghi | 64.5 | (31.6) | 9681 | (1430) | 35.0 | (12.8) | 9.2 | (0.9) | 35.4 | (8.8) |
| north | Kaleh ghochi | 79.5 | (28.1) | 6033 | (1054) | 27.4 | (6.7) | 6.1 | (0.8) | 23.0 | (4.4) |
| south-central | Kaleh ghochi | 13.1 | (7.0) | 7292 | (1592) | 15.5 | (3.7) | 15.9 | (2.9) | 22.8 | (8.5) |

Iran 2000

| | | | | | | | | | | | |
|---|---|---|---|---|---|---|---|---|---|---|---|
| central | Fandoghi | 27.7 | (31.6) | 8063 | (1797) | 31.6 | (21.6) | 9.7 | (1.9) | 33.0 | (11.1) |
| central | Fandoghi | 27.2 | (19.6) | 6622 | (1583) | 29.6 | (10.4) | 8.5 | (2.0) | 27.0 | (11.7) |
| central | Kaleh ghochi | 33.6 | (5.8) | 5534 | (742) | 24.0 | (6.0) | 15.0 | (1.4) | 16.2 | (3.3) |
| south-central | Kaleh ghochi | 26.5 | (9.9) | 8323 | (1157) | 25.3 | (5.5) | 6.0 | (0.7) | 36.9 | (13.0) |
| south-central | Kaleh ghochi | 46.6 | (21.7) | 6959 | (1604) | 27.7 | (9.2) | 6.2 | (0.9) | 24.3 | (7.5) |

Turkey 20001

| | | | | | | | | | | | |
|---|---|---|---|---|---|---|---|---|---|---|---|
| east | Siirt | 17.3 | (2.3) | 6946 | (1345) | 6.7 | (5.2) | 6.8 | (2.5) | 17.1 | (4.1) |
| central | Siirt | 14.7 | (2.2) | 6847 | (844) | 1.4 | (1.0) | 6.3 | (0.9) | 20.9 | (3.6) |
| central | Siirt | 20.7 | (3.3) | 5148 | (822) | 24.5 | (11.6) | 6.6 | (1.1) | 18.8 | (6.7) |
| central | Keten gomlegi | 11.9 | (3.3) | 7174 | (1141) | <1 | (na) | 4.3 | (1.0) | 26.9 | (7.1) |

United States

| | | | | | | | | | | | |
|---|---|---|---|---|---|---|---|---|---|---|---|
| CA 2000 | Kernan | 18.4 | (9.5) | 7401 | (2045) | 5.7 | (1.0) | 5.8 | (1.9) | 27.3 | (10.6) |
| CA 2001 | Kernan | 15.0 | (5.0) | 6654 | (1944) | 2.0 | (1.7) | 4.1 | (1.5) | 23.6 | (7.4) |

Beryllium, barium, titanium, and zirconium were typically near or below detection limits. Strontium had the largest concentration difference within the geographic regions and samples tested. All Iranian pistachio samples, all regions and all varieties, had high strontium concentrations relative to the other geographic samples analyzed. Generally, Iranian pistachios had strontium concentrations >20 μg/g. One set of samples from Turkey also had high strontium, but the other Turkey samples and all of the California samples had strontium concentrations near or below the detection limit. Iran and Turkey are two of the top four producers of strontium, which is mined as Celestine (SrSO₄). Although there are anthropogenic sources of strontium, it might be reasonable to expect strontium uptake in pistachios grown in regions of strontium production and export.

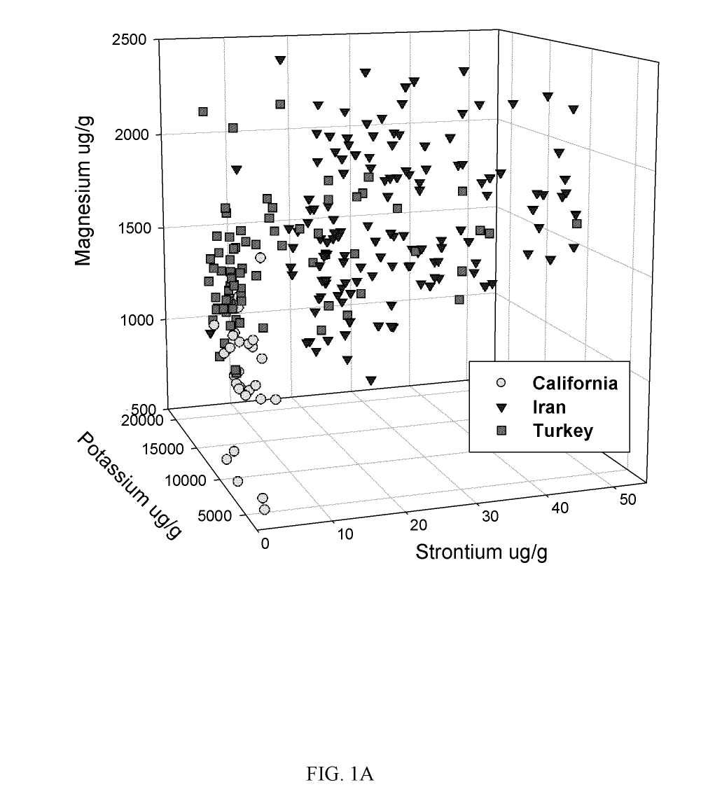

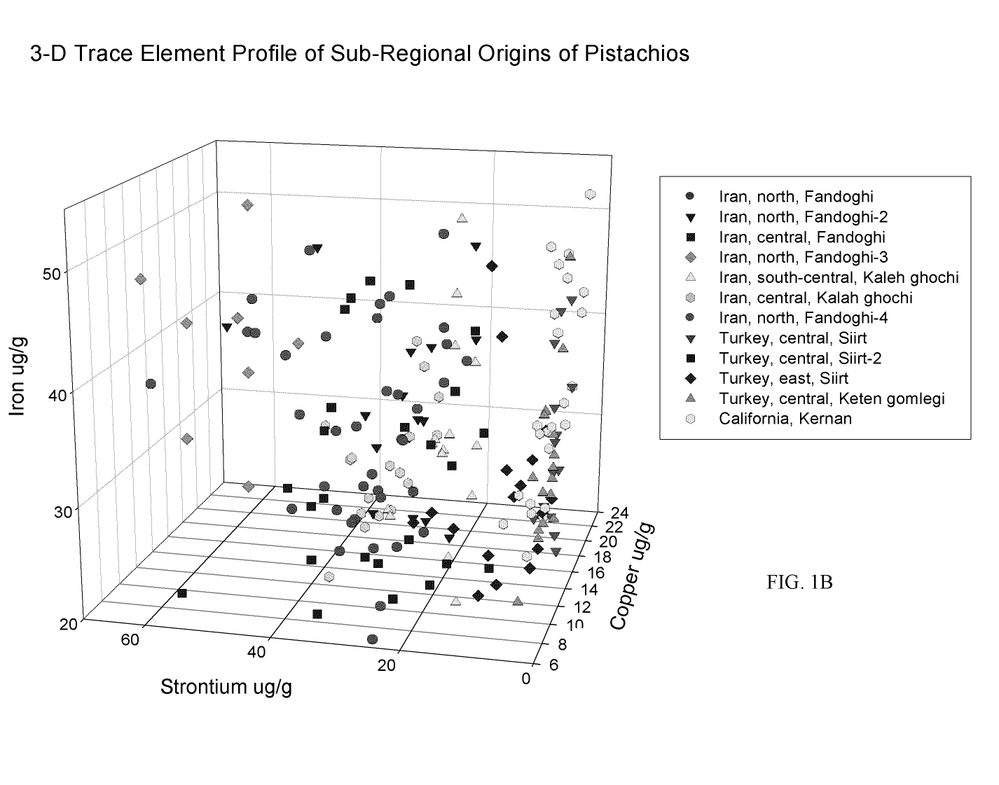

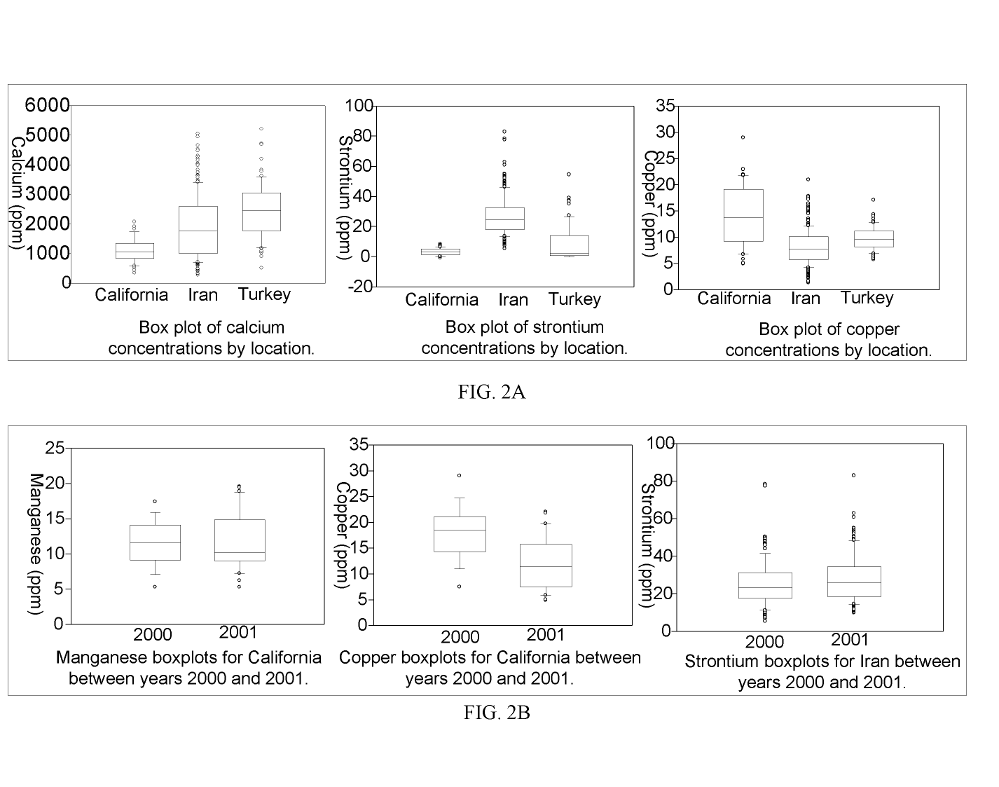

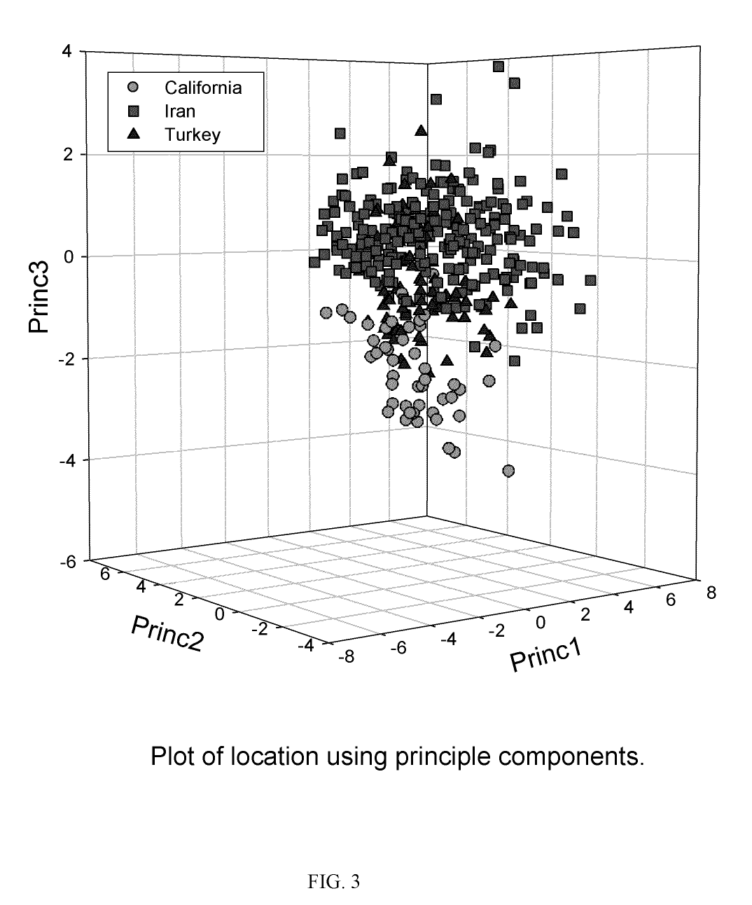

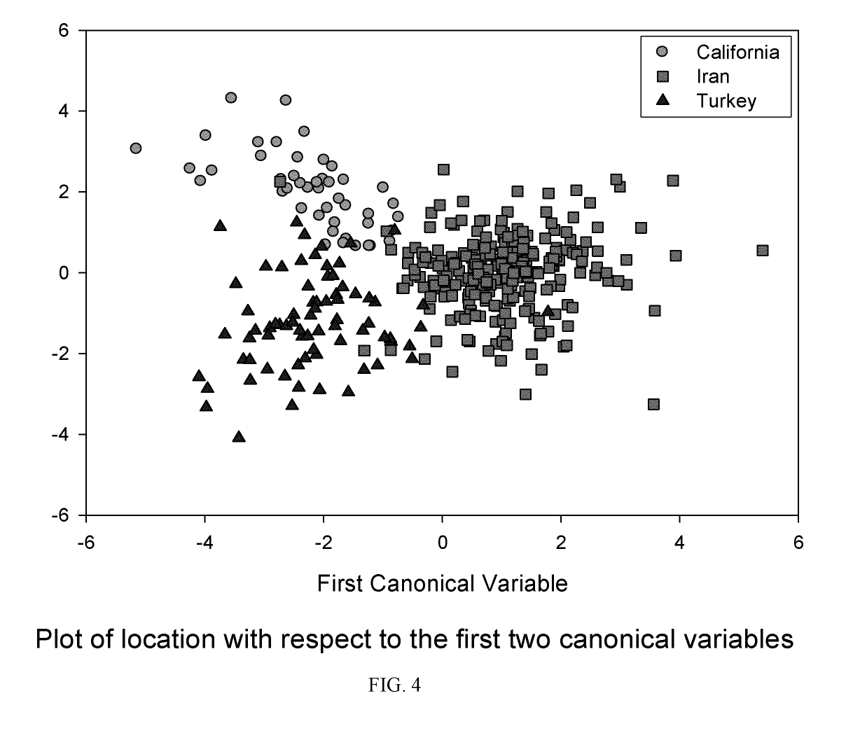

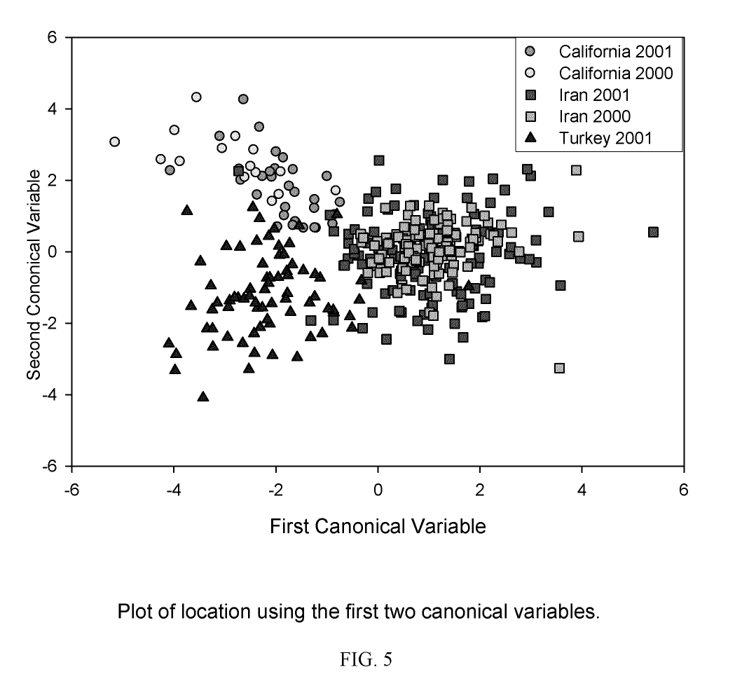

Interactions between strontium and calcium in plants are complex. Although strontium may compete with calcium, strontium usually cannot replace calcium in biochemical functions. Calcium-to-strontium ratios have been proposed by some authors for better understanding of source and uptake of cations (Kabata-Pendias and Pendias Trace Elements in Soils and Plants; CRC Press: Boca Raton, Fla., 1992). The calcium/strontium ratios in the samples tested, however, did not provide any additional discriminating power beyond simply comparing strontium concentrations. Unlike previous studies, the plant macroelements, calcium, potassium, magnesium, and phosphorus, had some differences based on geographic growing area, although singly these data have limited applications. The Californian samples generally had calcium concentrations a factor of 2-3 less than Iranian or Turkish pistachios. Within the 2001 season, for the geographic regions and samples tested (n=270), calcium varied by about a factor of 3.5 between geographic regions. Potassium typically was lowest in the Californian samples, varied from a factor of 0.3 to 2 lower than Iranian or Turkish samples. Potassium generally was highest in Iranian pistachios, although some subregions and varieties had lower potassium concentrations (discussed in more detail below). Magnesium typically was lowest in Californian samples from a factor of 0.5 to 2 less than Iranian or Turkish pistachios. Magnesium generally was highest in Iranian pistachios, for the 2001 season. Phosphorus varied by a factor of about 2 among all geographic regions tested. From a three-dimensional plot of potassium, magnesium, and strontium, one can see that geographic origin begins to separate in comparisons; see FIG. 1A and FIG. 1B. However, individually, and with reference solely to disclosed working embodiments, none of these elements alone appears to have discriminating power for the geographic regions tested. For examples, see a selection of box plots in FIG. 2A and FIG. 2B.

The plant microelements copper, iron, manganese, vanadium, and zinc also have some discriminating power with the geographic regions tested. More sophisticated computational analysis indicates that these data have value increasing modeling success, discussed below. Although individually no element was diagnostic of origin in disclosed embodiments, FIG. 1A and FIG. 1B illustrate by combining elements that there is better discrimination among some geographic regions. For example, Table 1 illustrates that copper ranged from 7 to 13 μg/g in the 2001 pistachios tested (n=270); over two seasons (n=371), copper ranged from 6 to 13 μg/g. Both the lowest and the highest copper concentrations occurred in Iranian pistachios in the same variety but indifferent subregions in Iran. Iron ranged from 24 to 48 pg/g, a factor of 2 difference between geographic regions for the 2001 season. Manganese ranged from 9 to 15 μg/g, a factor of only 1.5 difference between geographic regions. Vanadium for the 2001 season pistachios ranged from 4 to 21 μg/g. The highest vanadium concentrations were in Iranian pistachios, and the lowest vanadium concentrations were in Turkish and Californian pistachios. Zinc concentrations for the 2001 samples ranged from 37 to 37 μg/g. Generally, the highest zinc concentrations were found in Iranian pistachios while the lowest concentrations were found in pistachios from Turkey. For example, from a three-dimensional plot of strontium, iron, and copper, one can see that origins are beginning to separate (FIG. 1B). With more dimensions and modeling, better separations are possible.

FIG. 2A and FIG. 2B provide a selection of box plots. A variance analysis was performed, and in all cases the location group means were found to be different. Some interesting differences by groups were discerned visually by looking at the box plots. However, again, the distributions do overlap and it is difficult to determine a clear-cut rule for group classification from this analysis alone.

Another important result of the element concentration distribution is that no one region is responsible for all of the high or low concentrations. For example, Iranian pistachios had the highest average calcium, potassium, magnesium, strontium, vanadium, and zinc concentrations, while Californian pistachios had the highest average copper concentration. Turkey had the lowest iron, manganese, and zinc, and California had the lowest calcium, potassium, magnesium, and strontium. Overall, with so many differences, computational modeling as applied to elemental concentrations was a powerful tool.

2. Seasonal Variability

Seasonal variability also was investigated. A select group of box plots comparing the distributions of each element by season (for a given region) is shown in FIG. 2A. Analysis of variance was carried out, and seasonal group means differed. In general, the trace elements were lower in 2000 than 2001. Although the same geographic regions (Iran and California) and many of the same subregions (Iran-central and south-central) (see Table 1) were sampled in both the 2000 and the 2001 seasons, the exact same farms/trees were not systematically resampled; therefore, geographic differences may still contribute to differences observed between seasons. Strontium, the most discriminating element, was similar between seasons. Iranian pistachios in 2000 were >25 μg/g and in 2001 were generally >25 μg/g, while both Californian seasons (2000 and 2001) were <6 μg/g. Average calcium concentration in Iranian samples for both seasons was ≧1100 μg/g. Californian pistachios were ≦1100 μg/g. Copper values in Iranian pistachios in 2000 and 2001 were all 9 μg/g, while in both Californian seasons, the copper values were ≧12 μg/g.

Other elements, such as zinc, vanadium, and magnesium, although less dramatic, were somewhat different between the seasons. Overall, the 2001 Californian element concentrations were lower than the 2000 element concentrations; see the box plot in FIG. 2A for an example typical of the data trends. In contrast, the 2001 Iranian element concentrations were higher than the 2000 element concentrations. These seasonal trends for California and Iran were consistent for all elements tested. In a previous study with over 2000 potato samples collected over several seasons, only small variations between seasons were noted. Without sufficient seasonal data to demonstrate otherwise, the generation of databases for each season is likely necessary to ensure good predictability of any model used routinely.

3. Geographic Origin

FIG. 1A and FIG. 1B provide information concerning the concentration of strontium, potassium, and magnesium versus geographic growing origin. All varieties and two growing seasons are shown (n=371). The three-dimensional trace element profile of regional origins of pistachios shown in FIG. 1B provides information concerning the concentration of strontium, copper, and iron for the 2004 season. Subregions and varieties are shown. Variety differences were difficult to interpret, although within the same region and subregion, pistachios of different varieties were also grown in different orchards so geographic differences in subregions still exist to some extent (FIG. 1B). The Fandoghi variety was collected from three farms in the north region of Iran in 2001 and two farms in central Iran in 2000. Keeping seasons separate, the 2000 Fandoghi were all quite similar for calcium, copper, iron, potassium, magnesium, sodium, phosphorus, strontium, vanadium, and zinc. The Fandoghi from the 2001 season, in northern Iran, had a larger variation among all elements for this variety. The Kaleh ghochi variety for the 2000 season was collected at two south-central Iranian orchards; about half of the elements tested were similar while the remainder showed small variations.

4. Multidiscriminant Analysis

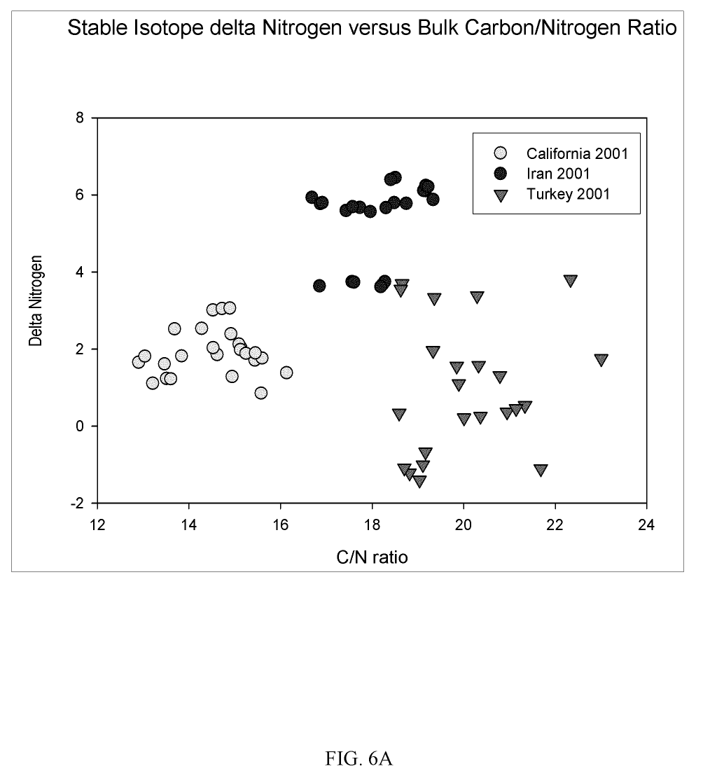

For initial data exploration, principal component analysis (PCA) was applied to the trace element data. A total of 372 pistachios samples representing three geographic regions with 20 samples from each were analyzed. A total of 270 samples were collected for the 2001 season from the three regions; see Table 1. The 2000 and 2001 element data were used for the computational analysis for certain disclosed embodiments. To adjust for different scales of measurement between trace elements, the data were normalized by subtracting the elemental means from each entry, and then each resulting difference was divided by the corresponding SD. Thus, each trace clement had an adjusted mean of zero and an adjusted SD of one. Sample scores with respect to the first three PCs are plotted in FIG. 3. Visual separation by growing region is not necessarily expected since PCs are measures of total sample variation and do not explicitly take into account variation between groups (locations) of interest. Some visual separation of samples from California and a combination of the samples from Turkey and Iran is observed however along the third PC. The first PC accounted for about 42% of the total variation. The second and third PCs accounted for about 17 and 14% of the total variation, respectively. The remaining seven PCs accounted for the remaining 27% of the total sample variation. For PC three, the most important elements were determined to be Sr, Fe, and Cu. For the second PC, the most important elements were K, Na, and Cu. The third PC, the most important elements were Mg, Mn, and P.

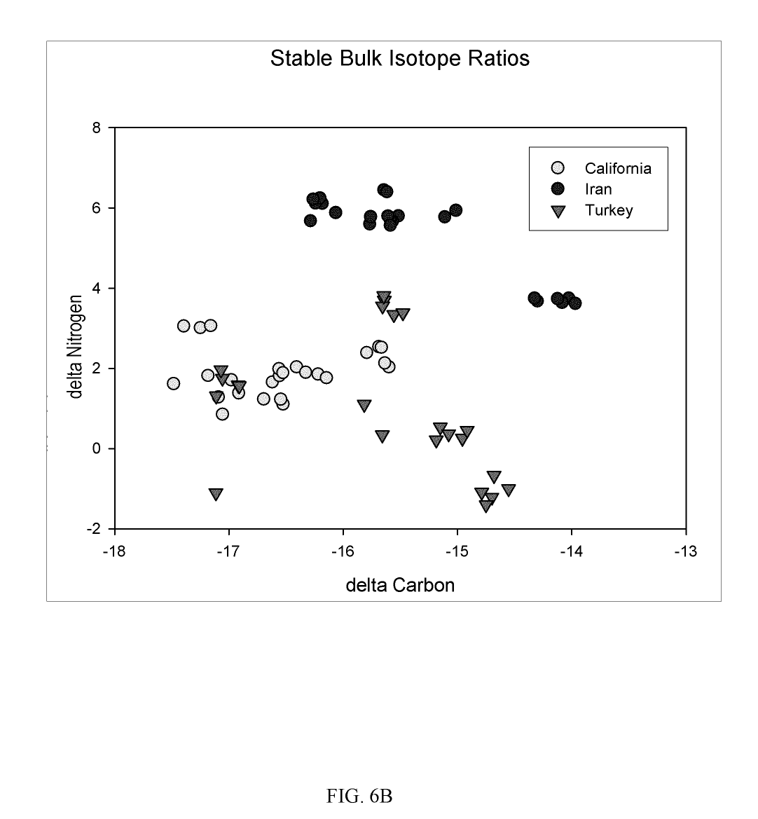

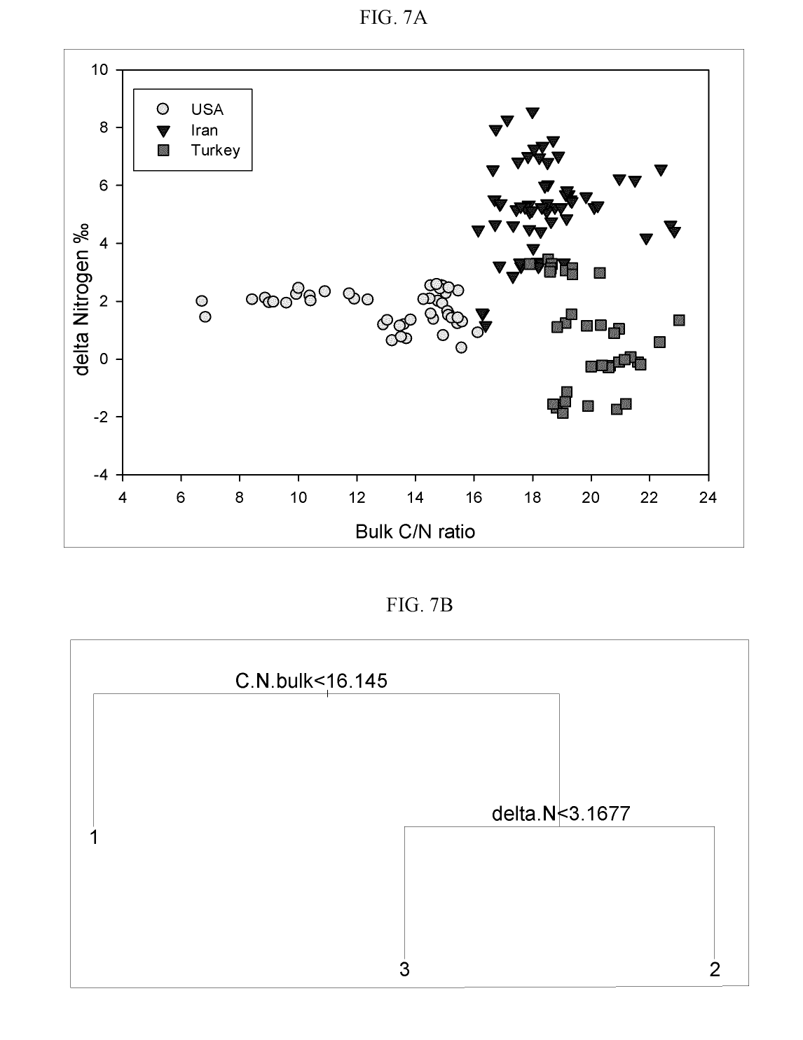

CDA was applied to pistachio data using the CANDISC procedure in the SAS software package. Because the number of groups was three, the total number of possible canonical variables was two. FIGS. 4 and 5 show scatter plots of the pistachio data using these two canonical variables. With reference to FIG. 4, there was good separation of the three regions using CDA. The three most important elements for the first canonical variable were Sr, Cu, and Na. The three most important elements for the second canonical variable were Ca, Fe, and Cu. FIG. 5 shows additional seasonal data, using the first two canonical variables.

Pattern recognition methods refer to methods that produce classification models based on the analysis of known sample data organized into predefined groups. Samples of unknown group membership then can be input into the model and assigned a probability of belonging to one of the predefined groups. Examples of these include the methods of linear and quadratic discriminant functions, non-parametric discriminant functions, and neural networks, to name a few. The methods have been discussed in earlier publications (Anderson et al., J. Agric. Food Chem. 1999, 47, 1568-1575, 26).

To get some sense of how well the prediction model will work on actual data, cross-validation was used. For cross-validation, the models are trained using all of the data minus one sample. This one sample is then presented to the model for classification. This process is repeated for each sample, and then the number of correctly classified samples is reported. Cross-validation results for this data set appear in Table 2.

TABLE 2
Cross-Validation Results and Percentage Correctly
Classified for Elemental Data Set
linear discrimination function

| | | 2001 data | |
|---|---|---|---|
| | | (trained on | |
| | all data | 2001 data) | all data with 25% |
| geographic | cross-validation | test set is | test set randomly |
| location | results (%) | 2000 data (%) | generated (%) |
| California | 97.83 | 65.50 | 100 |
| Iran | 95.22 | 84.87 | 93.55 |
| Turkey | 88.0 | not applicable | 88.89 |
| | | (no 2000 data) | |

Another approach, perhaps yielding a better assessment, can be accomplished by separating the data into training and test sets where the test set is larger than one sample. A reasonably large, say 25%, subset of the data is randomly selected for a test or validation set. A predictive model is developed using only the remaining data (called the training set). The test set is then presented to the model for classification.

First, all element data (both seasons) were used to develop a linear discriminant function using the DISCRIM procedure from the SAS® statistical software package. Other related methods, such as quadratic discrimination functions and nonparametric discrimination functions also were tried, but the linear discriminant function worked consistently better as measured by cross-validation and training/test set strategies. Neural network software also was applied yielding results similar to those obtained using linear discriminant function analysis. Cross-validation and test/training set results (percent classified correctly) for the linear discriminant function analysis are presented in Table 2. The ‘all data results’ in Table 2 demonstrate that a linear discriminant function model generalizes well to the “so-called” unknown (test sets) data from the made set of locations and seasons. Correctly classified cross-validations were >88% for all regions, while Californian pistachios were classified correctly with nearly 98% success. Errors in classification for Californian samples were most often confused with Turkish samples. The validation success was even better using a 25% test set; utilizing this approach, success rates were >89%, with Californian pistachios 100% successfully classified.

The “2001 data” results showed modest predictive ability when applied to the 2000 data. This maybe due to true seasonal differences, or perhaps a broader range of geographic locations was represented in the 2001 samples as compared to the 2000 samples. There were perhaps other unknown factors (e.g., variety) as well.

There are seasonal differences in the pistachios data. To further explore the consequences of this, the data were separated into training and test sets based on season. The training set consisted of all 2001 season data, and the test set consisted of all the 2000 season data. The results appear in Table 2.

### VIII. Using Stable Isotope Ratios of Elements for Determining Desired Information Concerning Food Products

A. Introduction

Plants and animals reflect characteristics of their environment and physiology through the stable isotope ratios of elements (e.g. ¹³C/¹²C, ¹⁵N/¹⁴N, ¹⁸O/¹⁶O and ²H/¹H) that form compounds in the organisms. Isotope ratios have been used in a chemical profiling method to determine geographic origin of biota (Guiseppe et al., ACS Symposium Series, 661,1997, pg 113-132, Kreuzer-Martin et al., PNAS, 2003,100,3, 815-19). Chemical, physical, and biological processes can have significant isotope fractionations. Stable carbon isotope methods use distributions of isotopes in organic matter that are a function of photosynthetic fixation, temperature, plant type (e.g., C3 v C4 plants) (Whilte et al., J. AOAC INTERNATIONAL, 1998, 81, 3, 610-618), and/or the environment (e.g., latitude) (Guy and Holowachuk, Can. J. Bot., 2001, 79, 274-283). For example, the ¹³C/¹²C ratios vary with geography and climate. Depending on the plant type (e.g. C3 or C4), each photosynthetic pathway discriminates differently against the heavier carbon isotope present in atmospheric CO₂. In addition, plants in humid environments, for instance, take in more CO₂; and therefore develop a lower ratio of ¹³C to ¹²C than plants in drier environments. Many chemical processes affect nitrogen isotopic composition, such as de-nitrification and mineralization. Climate and ecosystem variations, such as soil types, annual temperatures, and precipitation have been reported to affect nitrogen isotope ratios. Some geographical spatial variability in foliar nitrogen isotope ratios has been observed. Variation of the nitrogen isotope ratios varied from 3-15‰ relative to a small geographic region (Garten et al., Ecology, 1993, 74: 2098-2113). The range of nitrogen isotopic ratios was reported to reflect the spatial variability in atmospheric versus soil bioavailable nitrogen (Kendall and McDonnell Tracing Nitrogen Sources and Cycling in Catchments; Elsevier Science B. V.: Amsterdam, 1988, 519-576).

Processes affecting nitrogen isotopic composition include N-fixation, assimilation (e.g., uptake of ammonium, nitrate, etc.), mineralization, nitrification, volatilization, sorption/desorption, and denitrification. Across a broad range of climate and ecosystem types, soil and plant δ¹⁵N values systematically have been reported to decrease with increasing mean annual precipitation and decreasing mean annual temperature (Amundson et al., Global Biogeochem. Cycles 2003, 17(1), 1041). Globally, plant σ¹⁵N values are more negative than soils, suggesting a systematic change in the source of plant available N (organic/NH₄+ versus NO₃−) with climate (Amundson et al., Global Biogeochem. Cycles 2003, 17(1), 1041). Spatial variability in foliar δ¹⁵N has been observed within forested catchments (Garten, Ecology 1993, 74, 2098-2113). A compilation of data for nonfixing trees showed a 3-15% range in values among the same species relative to small geographic areas (Garten, Ecology 1993, 74, 2098-2113). The large range in δ¹⁵N reflects spatial variability in the relative amounts and bioavailability of atmospheric versus various soil sources of N (Kendall and McDonnell Tracing Nitrogen Sources and Cycling in Catchments; Elsevier Science B. V.: Amsterdam, 1988, 519-576). Carbon and nitrogen isotopes were determined and are reported as δ¹³C measured as CO₂ and δ¹⁵N measured as N₂. The total carbon/nitrogen ratios were also measured, and they are different for the three regions tested (n=71).

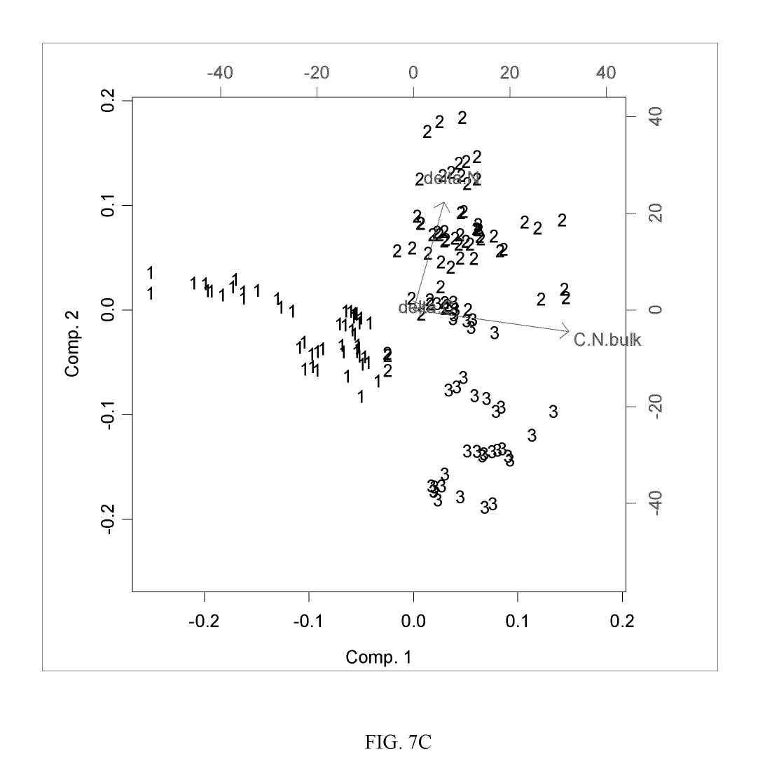

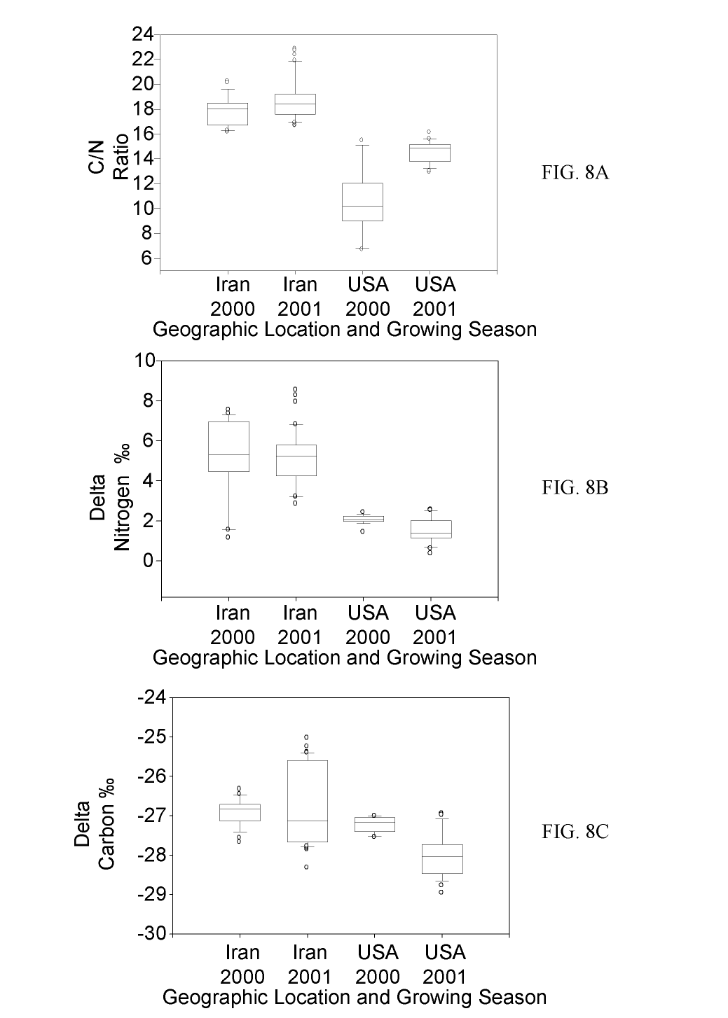

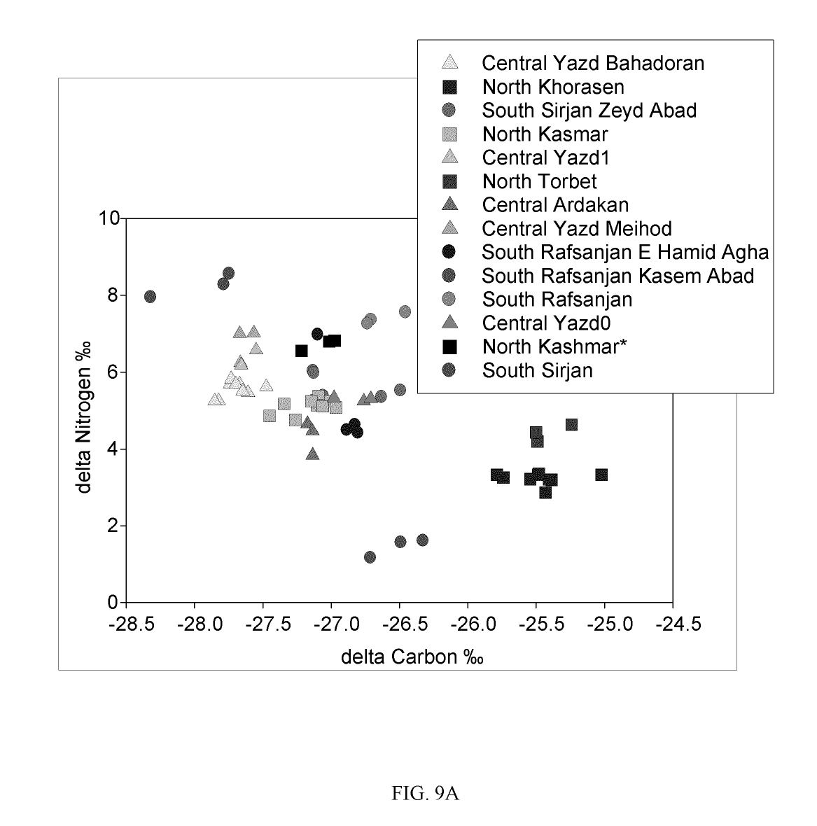

FIG. 6A shows the C/N ratio versus δ¹⁵N, and illustrates separation between the geographic regions. The δ¹³C and δ¹⁵N were evaluated, and there is some separation based on geographic region (FIG. 6B).

The grouping of the five Turkish samples with smaller (−17) δ¹³C values as compared to other Turkish samples was from the same variety and from the same region (e.g., Turkey, central, Siirt). The grouping of five Iranian samples with a larger value δ¹³C (−14) as compared with the other Iranian samples was also from a single variety and region (e.g., Iran, north, Fandoghi). Samples from all groups were rerun, and they strongly duplicated within the groups shown, including the five sample groups discussed above. The δ¹³C are apparently highly selective to the growing regions and conditions.

B. Regional Isotope Ratio Analysis Bulk nitrogen and carbon isotopes are determined by any suitable method, such as the process described in working embodiment provided by Example 2, and reported as δ¹⁵N‰, measured as N₂, and δ¹³C‰, measured as CO₂, and total bulk carbon/nitrogen ratios are calculated for the three regions tested. One working embodiment had n=146. For this working embodiment, the mean values of the C/N ratios, δ¹⁵N‰, and δ¹³C‰, found for pistachio samples grown in three different countries are from USA and Iran were statistically different for all three parameters (C/N, δ¹⁵N‰, and δ¹³C‰, p-value <<0.0001). USA and Turkey pistachios also were statistically different for all three parameters (p-value <<0.0001, unpaired t-test). Iran and Turkey pistachios were statistically different for C/N and δ¹⁵N‰ (p-value <<0.0001). However, the δ¹³C‰ was not statistically different (p-value=0.577, unpaired t-test) between Iran and Turkey pistachios.

Unlike many other chemical profiling techniques used to differentiate geographic origin where pattern recognition methods are required to make group separations, here a simple plot of bulk C/N versus δ¹⁵N‰ provides excellent group separations of the three countries (FIG. 7A). The separation by country is all the more notable since the data set included 2 growing seasons and several pistachio varieties. Tree-based models or algorithms provide an alternative method for classification problems. A hierarchical algorithm of decision rules is shown in FIG. 7B, which is useful for prediction/classification of pistachios in this data set. Restricting the algorithm to 3 terminal nodes as shown results in a good prediction of the data set, with a misclassification error rate of <5%. Adding two additional nodes provides nearly perfect prediction of the data set.

As might be expected, principal component analysis performed as previously described provides good separation of the data. PC 1 and PC 2 account for 65 and 31% proportion of the variance respectively, a cumulative proportion of 96% (FIG. 7 C).

The δ¹⁵N‰ values for pistachio samples from Iran, Turkey and USA showed greater variability than the δ¹³C‰ values, and ranged from about −3 to about 10. Higher δ¹⁵N has been attributed to greater plant uptake of soil-dissolved inorganic nitrogen, while lower δ¹⁵N has been attributed to greater plant uptake of the low-δ¹⁵N atmospheric nitrogen (ammonium). The three geographic regions (Iran, Turkey and USA) were each statistically different from the others: Turkey δ¹⁵N‰ pistachio values typically ranged from about −2 to about +3.0; USA δ¹⁵N‰ values ranged from about 0 to about +2.5; and Iran δ¹⁵N‰ values typically ranged from about +1 to about +9 (FIG. 7A).

Similar to δ¹⁵N‰, the bulk C/N ratios in pistachio samples displayed suitable variation for practicing the disclosed method, and values ranged from about 13 to about 23, specifically: Turkey C/N ratios typically ranged from about 18 to about 23; USA C/N ratios typically ranged from about 6 to about 16; and Iran C/N ratios typically ranged from about 16 to about 23. The bulk C/N ratio and δ¹⁵N‰ could be used to predict geographical origin for this 2 season, multi-variety, 3 country dataset. FIG. 7B.

Conversely, δ¹³C‰ values for pistachio samples from Iran, Turkey and USA showed modest variability and typically ranged from about −28.5 to about −24.5. USA and Turkey tended to have δ¹³C‰ values between −29 and −27, while Iran pistachio samples typically were −27.5 to −25. This range in δ¹³C‰ values is typical of other commodities, such as olive fruit (Bianchi et al., J. Agric. Food Chem. 1993, 41, 1936-1940, Angerosa et al, J. Agric. Food Chem. 1999, 47, 1013-1017. The modest range in δ¹³C‰ in olive fruit was attributed to the strict discrimination of the Calvin biosynthetic process. Even though there is only a modest range in δ¹³C‰, there is a statistically significant difference between USA and Iran/Turkey pistachios. This probably occurs because in addition to the plant discrimination process, there are environmental contributions to isotope discrimination. Values of δ¹³C‰ have been used to examine environmental variation, including water, latitude, and elevation effects. Guy and Holowachuk found that δ¹³C‰ values decreased (were more negative) with increasing rainfall. The USA pistachios were statistically different and more negative than pistachios from Iran or Turkey. This may indicate that USA samples experienced more moisture (more rainfall/irrigation water) during the study period. Both Iran and Turkey pistachios are grown in high elevation plains where there may be less available moisture. In addition to rainfall, values of δ¹³C‰ also have been correlated with latitude; however, there is little latitude difference between Iran, USA and Turkey, and the δ¹³C‰ were not associated with the small latitudes difference for the sub-regional sites within the study.

C. Seasonal Isotopic Ratio Analysis

Seasonal variability also was considered, although samples in working embodiments for each season were not always from the exact same farms. However, general sub-regions were re-sampled. Two seasons were collected from Iran (n=63) and USA (n=47) (Table 3).

TABLE 3
Seasonal bulk stable isotope values and standard deviations (±1
SD) dry weight for 2000 and 2001 Iran and USA pistachios

| Country/ | | Bulk C/N | δN‰ | δC‰ |
|---|---|---|---|---|
| Season | n | Avg (SD) | Avg (SD) | Avg (SD) |
| Iran 2000 | 23 | 17.85 ± 1.13 | 5.13 ± 1.81 | −26.89 ± 0.348 |
| Iran 2001 | 40 | 18.78 ± 1.62 | 5.20 ± 1.41 | −26.86 ± 0.98 |
| USA 2000 | 18 | 10.64 ± 2.52 | 2.09 ± 0.22 | −27.22 ± 0.18 |
| USA 2001 | 29 | 14.60 ± 0.84 | 1.53 ± 0.64 | −28.01 ± 0.56 |

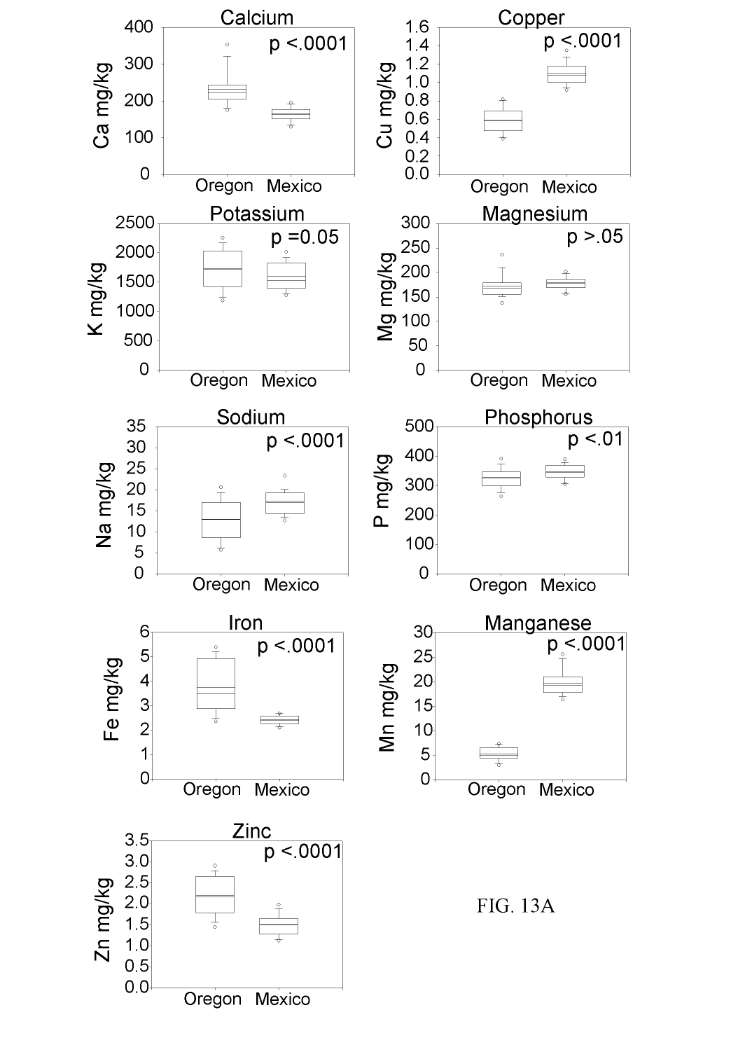

The C/N ratio, δ¹⁵N‰, and δ¹³C‰ were not statistically different for the two seasons in Iran (p-value=0.02, 0.8, and 0.9, respectively) (FIGS. 8A-8C). There were, however, seasonal differences in USA pistachio samples (FIGS. 8A-8C). The USA C/N ratio, δ¹⁵N‰, and δ¹³C‰ were statistically different, (p-value <<0.0001, 0.0001, and 0.0001, respectively). The USA pistachios' δ¹³C‰ values were more negative for the 2001 season. The average 2001 annual rainfall for this region was ca. 10% higher than 2000, consistent with the isotope trend. However, there is little literature to indicate that the magnitude of variation of δ¹³C‰ was based on seasonal moisture differences. The modest difference in rainfall coupled with irrigation would seem to be a tenuous association. In addition, since all of the ratios are statistically different, rainfall/irrigation alone is unlikely to account for the observed differences, although general climatic environmental differences do influence isotope ratios. Alternatively, USA pistachio results could indicate that there are sub-regional differences. A larger database could confirm these results. Although all USA samples were from California, the exact same farms/trees were not systematically sampled and the differences seen may be due to seasonal environmental effects or to sub-regional differences. It appears from this dataset that to confirm the effects or lack thereof of seasonal impact to geographic isotopic chemical profiling methods additional seasonal samples should be analyzed. Overall, however, the magnitude of the seasonal difference in USA isotopic values is small compared with the other geographic regions tested. Therefore, it does not adversely affect the isotopic geochemical profiling method. For example, the 2001 USA pistachio samples (n=29) were predicted with 100% success when using a tree model generated from the other samples (Iran, Turkey and 2000 USA, n=117).

D. Sub-Regional Isotopic Ratio Analysis

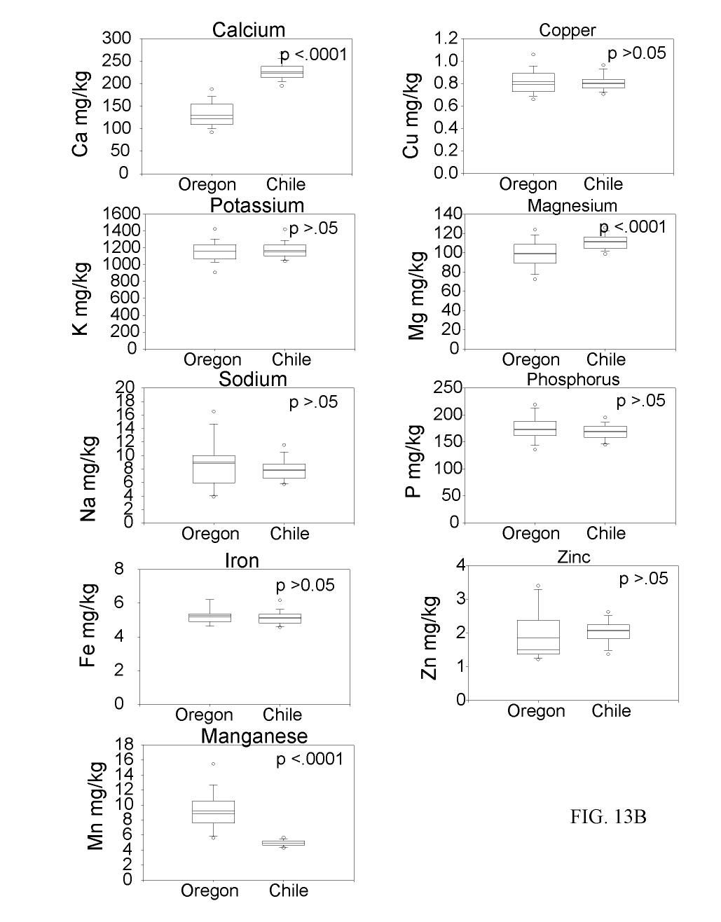

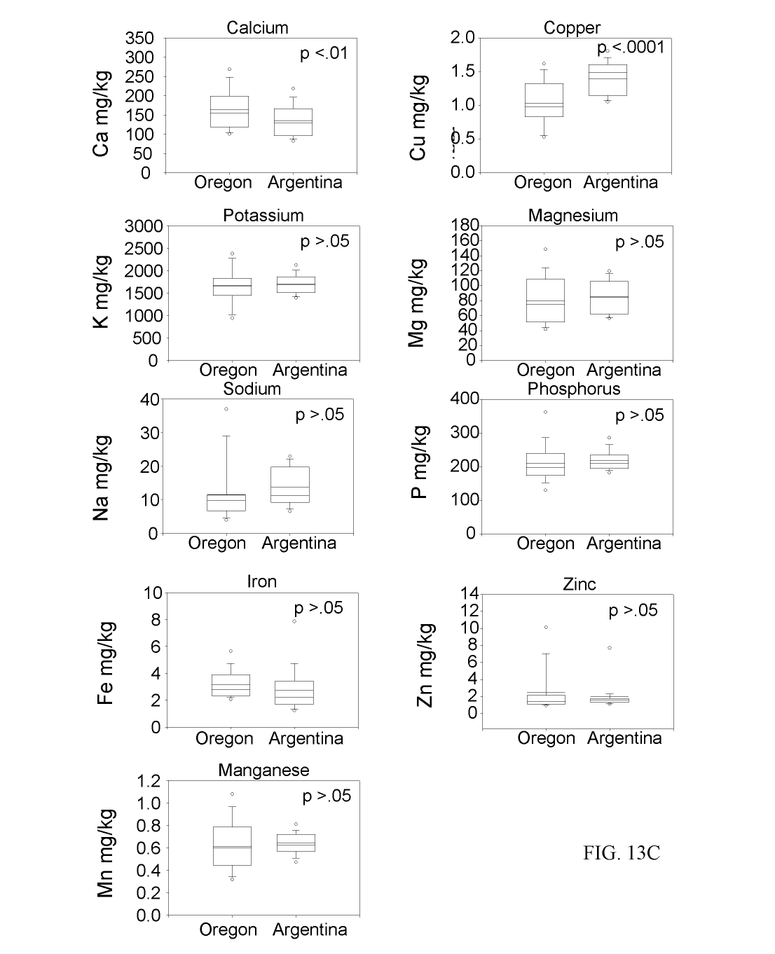

Regional differences also can be determined for food products using the disclosed embodiments of the present method. This embodiment is exemplified again by reference to pistachios. Regional pistachios were subdivided first into sub-regional units and then further subdivided into sub-locations. Three sub-regional growing areas were recognized for Iranian pistachios: North, Central, and South. Iran and Turkey sub-regional units were found to have some modest clustering character based on isotopic ratios (FIGS. 9A-9B). Iran pistachio samples from each sub-location, however, were strongly clustered based on the isotopic ratios (FIG. 9A). Nearly all of the pistachios were re-sampled, and analyzed so clusters are representative of the pistachio isotopic values and they are not an artifact of the analysis or analytical bias. Although sub-locations cluster, the general sub-regional growing areas do not exclusively cluster by sub-region (i.e. north, central, south). This is also true for the Turkey sub-regional units, Central and East, which are not as strongly clustered in their small sub-location units. See FIG. 9B. Therefore, one could not a priori predict the isotopic ratios based on sub-regional designations. This appears to be an important caveat of authenticity research that, without an adequate fully representative database, predictions should be made prudently.

Development of tree model classification algorithm while withholding specific sub-regional samples still results in excellent geo-locating success. For example, an algorithm developed with USA, Turkey and Iran samples (n=124) minus all samples from the northern Iran region had a >98% success rate of the training data set. This algorithm was then used to classify the northern Iran samples (n=25) and it had a 100% success rate. Similarly, success rates were achieved with various combinations of training/predicting datasets of sub-regional pistachio isotope samples. Therefore, within this data set, although there are some sub-regional differences relative to the overall isotopic differences between the three regions, the sub-regional differences are small and do not adversely affect geo-locating success.

E. Species Variety Differences by Isotopic Ratio Analysis

The differences in food product varieties also can be determined using the disclosed method. This embodiment can be exemplified by reference to working embodiments that used pistachio varieties. Two varieties from Iran were analyzed: Fandoghi and Kaleh Ghochi (n=63). Two varieties from Turkey also were analyzed; Sliirt and Keten Gomlegi (n=36) (Table 4).

TABLE 4
Variety differences in bulk stable isotope ratios
and standard deviations (±1 SD) dry weight

| | | | Bulk C/N | δN‰ | δC‰ |
|---|---|---|---|---|---|
| Country | Variety | N | Avg (SD) | Avg (SD) | Avg (SD) |
| Iran | Fandoghi | 36 | 18.18 ± 0.90 | 4.93 ± 1.20 | −26.89 ± 0.87 |
| Iran | Kaleh | 27 | 18.78 ± 2.05 | 5.49 ± 1.91 | −26.84 ± 0.71 |
| | Ghochi | | | | |
| Turkey | Sliirt | 27 | 19.79 ± 1.11 | 0.47 ± 2.02 | −27.14 ± 1.05 |
| Turkey | Keten | 9 | 20.51 ± 1.43 | 1.12 ± 0.27 | −26.45 ± 0.24 |
| | Gomlegi | | | | |

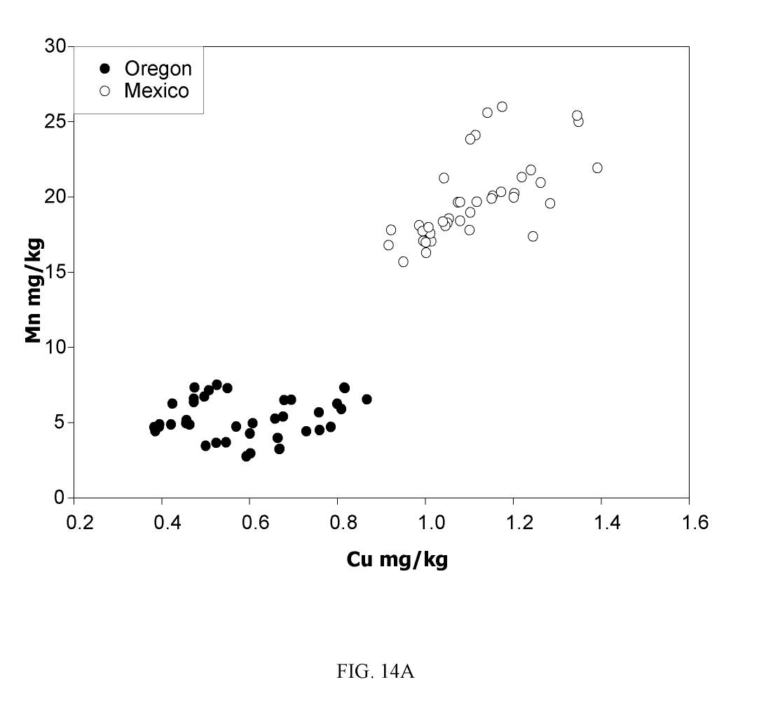

The C/N, δ¹⁵N‰, and δ¹³C‰ for the two Iranian varieties were not statistically different (p-value=0.12, 0.16, and 0.81, respectively, student's unpaired t-test). The C/N, δ¹⁵N‰, and δ¹³C‰ for the two Turkey pistachio varieties were not statistically different (p-value=0.13, 0.35, and 0.06, respectively, student's unpaired t-test). The pistachio varieties do not separate readily, as seen in FIG. 10, as a function of variety only, though embedded in such an analysis is variation of growing area since there were no different varieties from adjacent pistachio trees. As compared to geographic differences, variety does not appear to affect the isotopic differences seen within this dataset.

Models developed without a specific variety were still able to successfully classify the geographic origin, as might be expected since there is no statistical difference between varieties within a geographic region. For example, a tree model developed with USA, Iran, and only Keten Gomlegi Turkey pistachios (n=119) was able to successfully (100%) classify Turkey Sliirt samples (n=27).

### EXAMPLE 1

### This Example Describes a Procedure for Chemical Analysis and Stable Isotope Ratio Analysis of Pistachios

Chemicals and reference materials used in certain of the examples were obtained as follows: concentrated nitric acid, trace element analysis grade (J. T. Baker, St. Louis, Mo.); elemental stock standard solutions (J. T. Baker): certified reference materials (CRM); NIST 1575 Pine Needles; NIST Oyster Tissue 1566a; NIST Rice Flour 1568a; NIST 1577b Bovine Liver; NIST 8433 Corn Bran (National institute of Standards and Technology, Gaithersburg, Md.); and NRC TORT-2 Lobster Hepato-pancreas (National Research Council Canada, Institute National Measurements Standards, Ottawa, Ontario, Canada).

The inductively coupled plasma atomic emission spectrometer (ICPAES) was equipped and setup as follows: Varian model (Palo Alto, Calif.) Liberty ISO ICPAES; PMT, 650 V; nebulizer, 85 psi; auxiliary, 1.5 L/min; pump rate, 13 rpm; two integrations: 1.0 scan integration time; acid flexible tubing, 0.030 am ID (internal diameter); wavelengths and background corrections have been previously presented (24, 25). A temperature controller/digester used was a Lab-line microprocessor digestor block and controller. The capillary electrophoresis (CE) was equipped and setup as follows: Hewlett Packard model (Palo Alto, Calif.) HP 3D CE: diode array detector, 50 μm ID×64.5 cm fused silica extended bubble light path capillary column; sample injection, 50 mbar; 2 s; applied voltage, −25 kV; capillary temperature, 16° C.: detection at 350 nm and reference at 225 nm. The analytical method time was 7 minutes.

Nitrogen (¹⁵N) and carbon (¹³C) stable isotopes were measured on a stable isotope mass spectrometer (MS) (Finnigan, MAT 251). Isotopic data use the standard isotopic notation (δ) in per mil ( 0/00) relative to the Pee Dee Belemnite (PDB) scale. Calibration to PDB was done using NBS-19 and NBS-20 standards of the National Institute of Standards and Technology (MD). External precision estimates of 15N and carbon 13C, based on replicate analysis of acetanilide and oxalic acid standards, were ±0.12% and 0.11% respectively.

Pistachio samples were collected on-site in Turkey and Iran and shipped directly to the laboratory. Chain of custody was maintained for all. California samples were provided by the California Pistachio Commission. Specific sub-regions cities, varieties, and season information were known for all samples analyzed. Each pistachio sample was analyzed as the whole nut (no shell). Samples were analyzed on a dry weight basis. For elemental analysis, pistachio samples were digested. A ca. 1.0 gram sample was taken, representing one nut, and the sample was digested with 3.0 mL of nitric acid (trace metal grade) in a 10 ml. graduated Kimax culture tube on a programmed heating block. The samples were allowed to react for ca. 4-8 hours in a hood at ambient temperature. Then, the samples were digested using a programmable heating block. The samples were ramped 140° C. over an hour and then maintained at 140° C. for 3-4 hours. Digestion was confirmed complete when no nitrous oxide gases were evolved.

The samples were diluted with type I water (18 Mohm cm) and mixed thoroughly using a vortexer. The samples were filtered through a 0.45 μm filter prior to analysis. Analysis was by ICPAES. For anions (inorganic and organic acids), pistachio samples were extracted. The background electrolyte used was 2,6-pyridine-dicarboxylic acid (PDC), and 0.5 mM hexadecyltrimethylammonium bromide was used with 5 mM PDC at a pH of 5.6. All samples were filtered prior to analysis through a 0.22 μm filter. Samples for isotope analysis were dried overnight at 60° C., ground to a fine powder, and loaded in capsules for MS analysis.

The chemical analytical technique is well-suited to analysis of modest-to-small samples. A minimum of at least as small as 500 mg can be used, although 1 gram samples were used in this example. Dilution factors are minimized here; only a factor of 10 as compared to typical digestions that involve dilution factors of 50 or more. This small dilution factor permits determination of additional elements that would otherwise be below instrument detection limits. In addition, as a pollution prevention mechanism, this technique uses fewer reagents and in small volumes; thus, this technique reduces waste.

To insure quality control, each analytical batch contained a minimum of 25% quality control samples, including check standards, duplicates, spikes, and CRMs. Each individual analyte was quantitated based on calibration curves consisting of 3-4 standard levels each, with correlation coefficients of >0.98. Over 50 CRM samples have been analyzed; CRMs were dominantly plant matrices where available. Typical percent standard deviation (% SD) was <10%, although analytes close to method detection limits had higher % SDs. Spike recoveries and cheek standards were typically within ±10% of their true value.

### EXAMPLE 2

### This Example Describes a Procedure for Stable Isotope Ratio Analysis of Pistachios

Nitrogen (δ¹⁵N‰) and carbon (δ¹³C‰) stable isotopes and bulk C/N ratios were measured on a stable isotope mass spectrometer (MS) (Finnigan MAT-251, ThermoFinnigan, Waltham, Mass.). Isotopic data use the standard isotopic delta notation (δ), in per mil (‰), relative to the Pee Dee Belemnite (PDB) scale for carbon isotopes and relative to air (¹⁵N) for nitrogen. By convention, the following equation for delta was used for carbon (and an analogous equation for nitrogen):

Delta(δ)¹³C‰=[(¹³C/¹²Csample)−(¹³C/¹²Cstd)/(¹³C/¹²Cstd)]×1000

Enrichment of heavy isotopes, relative to the standard, gives positive values, while enrichment of light isotopes, relative to the standard, gives negative values. Calibration to PDB was done using NBS-19 and NBS-20 standards of the National Institute of Standards and Technology (Gaithersburg, MD).

Samples from Turkey and Iran were collected on-site and shipped directly to the laboratory. Chain-of-custody was maintained for all samples. USA samples were provided by the California Pistachio Commission. Specific sub-regions/cities, variety, and season information was known for all samples analyzed. Each pistachio sample was analyzed as the whole nut (no shell). Samples for isotope analysis were dried overnight at 600° C., ground to a fine powder using a small coffee grinder (2 oz., Toastmaster, Boonville, Mo.), and loaded in capsules for MS analysis. The chemical analytical technique is well suited to analysis of modest to small samples; a minimum of 2.0÷0.5, mg can be used. A total of 146 pistachio samples were analyzed from USA, Iran, and Turkey (n=47, 63, and 36, respectively). Samples from two growing seasons were analyzed from two regions: Iran in 2000 (n=23) and 2001 (n=40) and USA in 2000 (n=18) and 2001 (n=29). Two pistachio varieties were analyzed from two regions, Iran Fandoghi (n=36) and Iran Kaleh Ghochi (n=27); and Turkey Sliirt (n=27) and Turkey Keten Gomlegi (n=9). In order to minimize any potential for day-to-day bias, samples from any designated group were typically analyzed in three different batches. Samples in each batch were re-sampled, ground, loaded in capsules, and analyzed on different days.

Each sample was analyzed in triplicate. NIST 8542 and NIST 8548 samples were also analyzed in each batch. External precision estimates of δ15N‰ and δ13C‰, based on replicate analysis of acetanilide and oxalic acid standards, were δ0.12‰ and ±0.11‰, respectively. Graphical presentations and t-test used SigmaPlot 2003 for Windows, Version 8.0, SPSS, UK, Ltd. Model results used S-Plus 2000, Lucent Technologies, Inc. The use of statistical and multidiscriminant analysis for characterizing geographic growing origin has been previously described.

### EXAMPLE 3

### This Example Describes a Procedure for Stable Isotope Ratio and Trace Element Analysis of Blueberries, Pears, and Strawberries

A. Materials and Methods

Chemicals and reference materials used in certain of the examples were obtained as follows: concentrated nitric acid, trace metal grade Fisher Optima (Pittsburgh, Pa.); elemental stock standard solutions, Alfa Aesar Specpure (Ward Hill, MA); and 18 MΩ·cm water (Barnstead, Dubuque, IA) were used.

The inductively coupled plasma argon atomic emission spectrometer (ICPAES) was used to analyze digested samples. The following parameters were employed: model, Liberty 150 ICPAES (Mulgrave, Victoria, Australia); V-groove nebulizer 85 psi; Varian SPS5 autosampler system; scan integration time, 1 sec (all elements); acid flexible tubing 0.030 mm ID (internal diameter); replicates, 3 (all elements); scan window, (1st order) 0.120 nm; photo multiplier tube voltage, 650 V; plasma flow, 15 L/min.; auxiliary flow, 1.50 L/min.; sample uptake delay, 13 sec.; pump rate, 15 rpm; instrument stabilization delay, 13 sec.; rinse time, 60 sec. The wavelengths selected were: Ca 214.434; Cd 422.673; Cr 267.716; Cu 324.754; Fe 259.94; K 285.213; Mg 257.61; Mn 231.604; Na 213.618; Ni 769.896; P 589; V 294.402; Zn 213.856.

1. Bulk Stable Isotope Analysis

Nitrogen (δ¹⁵N‰) and carbon (δ¹³C‰) bulk stable isotopes and bulk C/N ratios were measured and calculated on a stable isotope mass spectrometer (MS) (Finnigan MAT-251, ThermoFinnigan, Waltham, Mass.). Isotopic data use the standard isotopic delta notation (δ), in per mil (‰) relative to the Pee Dee Belemnite (PDB) scale for carbon isotopes and relative to air (¹⁵N) for nitrogen. By convention, the following equation for delta was used for carbon (and an analogous equation for nitrogen):

(δ)¹³C‰=[((¹³C/¹²Csample)−(¹³C/¹²Cstd))/(¹³C/¹²Cstd)]×1000

The enrichment of heavy isotopes relative to the standard gives positive values and enrichment of light isotopes relative to the standard gives negative values. Calibration to PDB was done through the NBS-19 and NBS-20 standards of the National Institute of Standards and Technology (MD).

2. Oregon Field Sampling

Oregon samples were collected in summer 2002 from field locations spanning the state (˜350 miles in length), including Hood River, Portland, Salem, Brownsville, Corbett, Corvallis, and Central Point depending on commodity. At each Oregon farm, approximately 7.6 L (8 quarts) of blueberries (Vaccinium caesariense/corymbosum), 7.6 L (8 quarts) strawberries (Fragaria ananassa), and >12 pears (Pyrus communis) were collected by hand and labeled according to farm location (sub-region) and variety.

All Oregon samples were hand picked at each individual field location, except for one pear collection site. Pears collected from the site labeled ‘Portland’ were purchased at a local organic food market where they were labeled as having been grown in the Portland area. Individual field replicates were analyzed separately and represent randomized field collection (picked from multiple blueberry bushes, strawberry rows, or pear trees). Only the most common varieties in the fresh market, both nationally and internationally, were analyzed. The international samples were collected from Oregon grocery stores that offered produce labels indicating geographic origin. Fresh market samples were intentionally collected when they would be out of “season” for Oregon, and therefore more likely from South America/Mexico. Based on the differences in availability of these fruits, the assumption was made that these internationally labeled samples were authentic. No international sub-locations were specified.

3. Sample Preparation and Analysis

All samples were rinsed under a stream of tap water, followed by a three-fold rinse with 18Ω·cm water, and blotted dry with paper towels. Each sample was homogenized using a Robot Coupe industrial BLIXER RS1 BX6 (Ridgeland, MS) and liquid nitrogen, until the homogenate resembled a fine powder. All samples were stored in individually HNO₃ cleaned glass jars at −20° C. until further analysis. Samples were processed according to a method previously described (Anderson, and Smith, J. Agric. Food Chem. 2002, 50, 2068-2075, Anderson and Smith, J. Agric. Food Chem. 2005, 53, 410-418, Anderson et al., J. Agric. Food Chem. 1999, 47, 1568-1575). Analysis of total elements within the digestate was performed using an ICPAES. This multi-element method, with the use of the ICPAES requires little sample (1g), and low solvent use. This leads to decreased reagent cost, less waste generated, decreased disposal cost, and fewer hazards to the analyst.

4. Isotope Analysis

Pear samples were analyzed as the whole pear from freeze fracture homogenization. Homogenates were freeze-dried. Samples were loaded in capsules for MS analysis. The chemical analytical technique was well suited to analysis of modest-to-small samples; a minimum of 2.0±0.5 mg was used.

5. Quality Control and Statistical Analysis

Certified Reference Materials (CRMs) were included in each multi-element analytical batch: NIST (National Institute of Standards and Technology) 1515 Apple leaf, NIST 1573a Tomato leaf (NIST, Gaithersburg, Md.). CRMs, check standards, and blanks accounted for at least 25% of each analytical batch. A minimum of three standards were used per calibration curve with R² values >0.99. Detection limits were calculated as three standard deviations based of seven blanks. Average recoveries for each element are as follows: Ca, 108%; Cu, 120%; Fe, 98%; K, 96%; Mg, 125%; Mn, 106%; Na, 99%; P, 120%; Zn, 116%. Check standard recoveries averaged 101%.

Each isotope sample was analyzed in triplicate. NIST 8542 Sucrose ANU-sucrose and NIST 8548 IAEA-N2-ammonium sulfate samples were analyzed with each batch. External precision estimates of δ¹⁵N‰ and δ¹³C‰, based on replicate analysis of acetanilide and oxalic acid standards, were ±0.12‰ and ±0.11‰, respectively.

Several statistical analysis methods were applied to the data. Multiple comparisons ANOVA were used in sub-regional and variety analysis by Sigma Stat for Windows, Version 2.0 (Systat, Point Richmond, Calif.). Graphical presentations and t-tests comparing geographical location used SigmaPlot 2003 for Windows, Version 8.0 (Systat, Point Richmond, Calif.). Significance was determined using a two sampled t-test in Sigma Plot. Canonical discriminant analysis (CDA), linear discriminant function, and quadratic discriminant function analyses were applied utilizing SAS version 2.0 (SAS Institute Inc., Cary, N.C.) and neural network and genetic neural network analysis using NeuroShell Classifier (Ward Systems Group Inc, V2.2, Frederick, Md.). Hierarchal tree model and principal components analysis results used S-Plus (Lucent Technologies, Inc.). The models were tested with up to three different approaches, re-substitution, cross-validation (i.e. leave-out-one) and by test-set. From each geographic group 5 samples were randomly selected (from 40) to form a test-set of 10 samples (2 geographic regions) and removed from the training set. The remaining samples (nominally 35 from each group, a total of 70) were used as the training set for the classification models. Once trained, each model was then used to classify the 10 “unknown” samples in the test-set. Variety testing was preformed only when two varieties at the same site were obtained so as not to have the confounding variation created by different geographic sites. Training and test-sets were created that were variety specific, test sets typically n=8. For example, a training set was created without a specific variety and the test-set contained only the variety withheld from the training set.

B. Regional Element Profiling

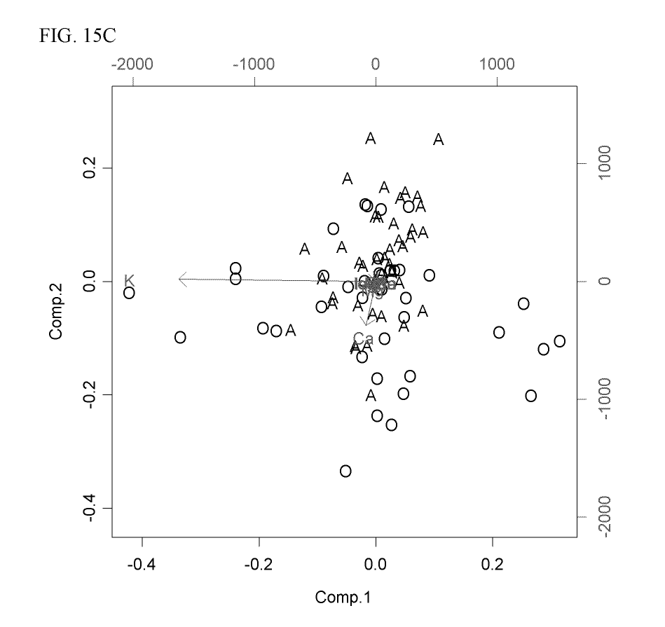

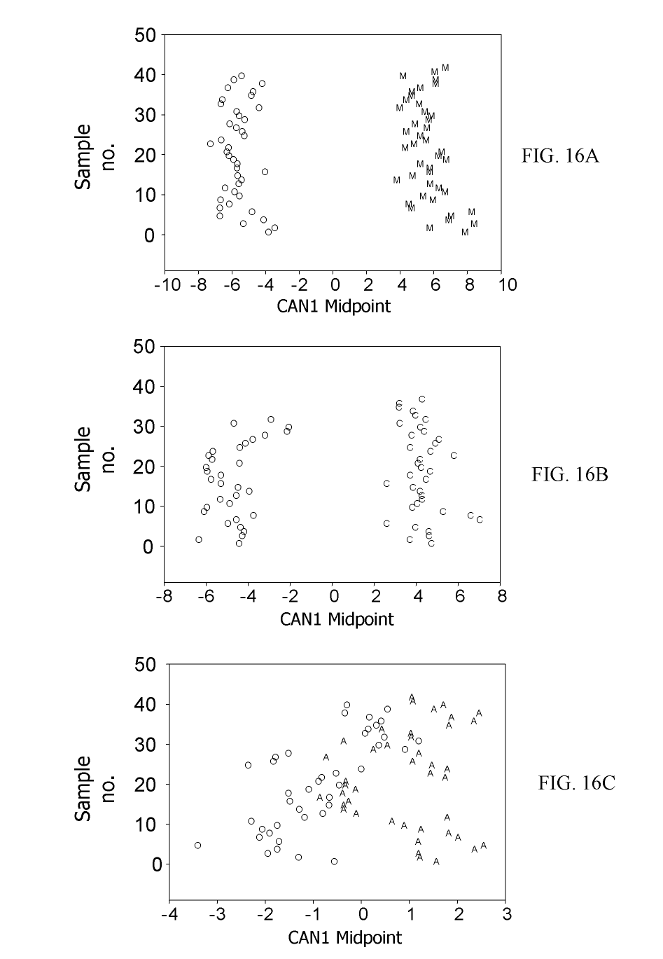

Nine elements were consistently above detection limit: calcium (Ca), copper (Cu), iron (Fe), magnesium (Mg), manganese (Mn), potassium (K), sodium (Na), phosphorus (P), and zinc (Zn). Cadmium (Cd), chromium (Cr), vanadium (V), and nickel (Ni) were often near or below detection limits. Box plots, FIG. 13A-13C, are shown for each fruit, the boundary of the box closest to zero indicates the 25th percentile, the solid lines within the box mark the mean and median, and the boundary of the box farthest from zero indicates the 75th percentile. Whiskers above and below the box indicate the 90th and 10th percentiles, while symbols represent the 5th and 95th percentiles. Significant differences were defined at the 95% confidence level. Simple elemental distribution plots shows clustering by geographic origin for Oregon and Mexican strawberries and Oregon and Chilean blueberries, FIG. 14A-14B.

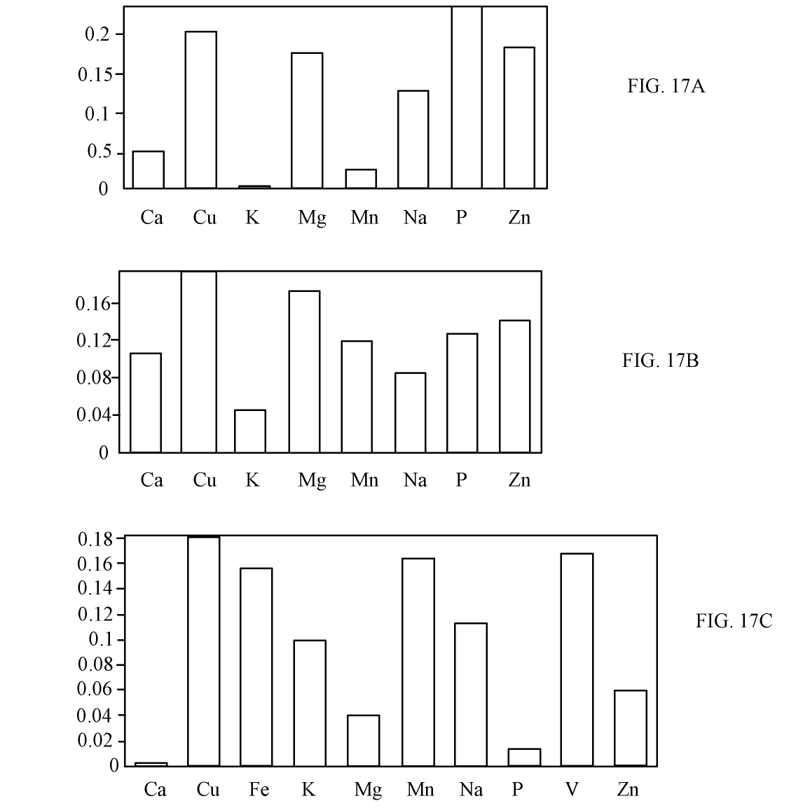

The data were further analyzed to explore the feasibility of classifying fruit samples according to geographic origin. Initially this is investigated through statistical visualization methods. Principle component analysis, measures variation in the elemental concentrations in the samples, but does not take into account group (geographic origin) membership; however, it is sometimes the case that a large percentage of the total variation can be explained by the first few principle components. This effectively reduces the number of variables needed to describe variation among samples. Principle component analyses (PCA) of geographic origin group memberships are well manifested for strawberry and blueberries, but not for pear, FIGS. 15A and 15B. Since PCA does not take into account group membership, to get the best possible view of group cluster canonical discriminant analysis (CDA) was used. Strawberry and blueberry separate well, although there is some separation, pears overall are still poorly separated by geographic origin group, FIG. 14A-14B. The data were modeled to further explore the feasibility of classifying fruit samples according to geographic origin; linear discriminate function, quadratic discriminant function, neural network, genetic neural network, and hierarchal tree modeling methods were employed, discussed below for each commodity.

C. Regional Strawberry Analysis (Oregon vs. Mexico)

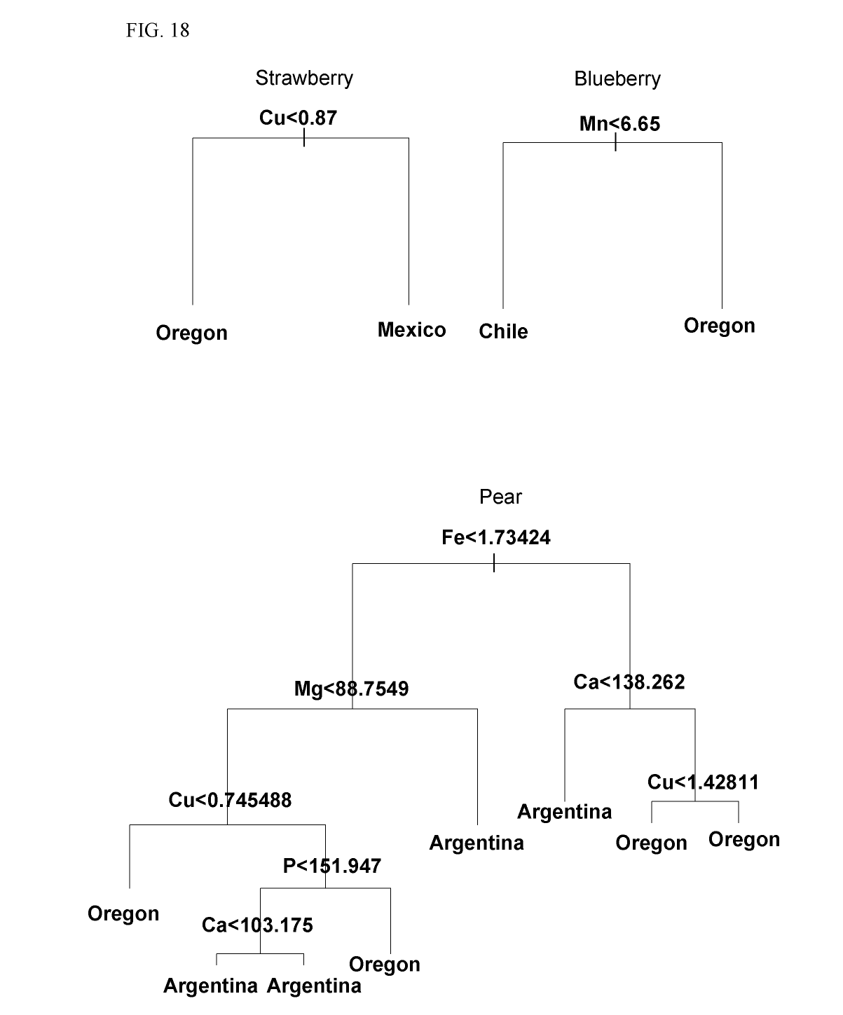

General element concentration variability in Oregon and Mexican strawberries is shown in FIG. 13A. Strawberry concentrations of Ca, Cu, Fe, Mn, Na, and Zn showed significant separation (p<0.0001), as did P and K concentrations (p<0.01, 0.05 respectively, 78 d.f.). No significant difference for Mg concentration was observed between Oregon and Mexican strawberries. Combinations of Ca, Mn, K, Cu, Fe, or Zn could be used to visually depict geographic origin group clustering, for example see FIG. 14A. Other elemental combinations were tried; however, these elements gave good visual separation of geographic origin groups. On average, Mexican strawberries contained 380% the concentration Mn and 190% the Cu concentration of Oregon strawberries, while Oregon strawberries, contained more Ca (29%), Fe (35%), and Zn (32%) than Mexican strawberries. Principal component analysis (PCA) generates principle components that are linear combinations of the original variables. The first principle component describes the maximum possible variation that can be projected onto one dimension. PCA on strawberries showed that the first three components accounted for 99.7% of the total variability (95%, 98%, 99.7% respectively). PCA and CDA showed strong visual clustering with Mexican and Oregon strawberries, (FIGS. 15A and 16A).

D. Strawberry Modeling

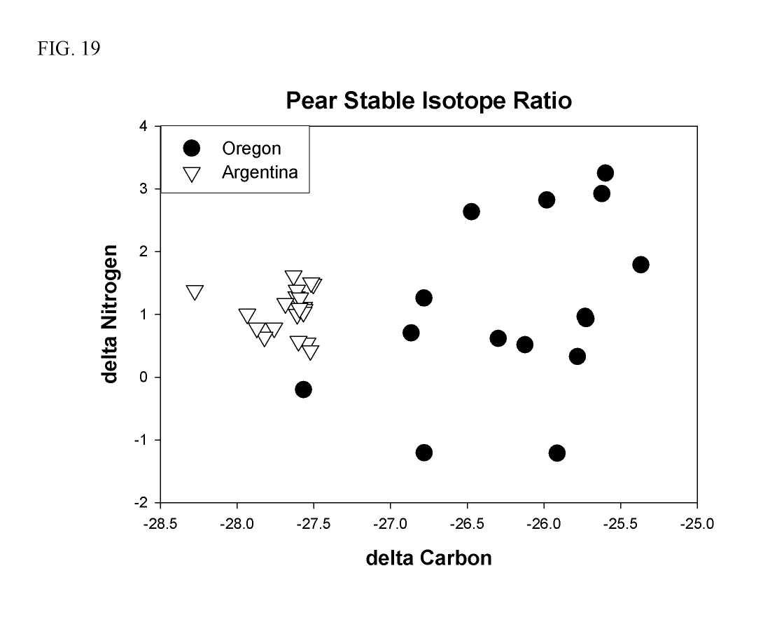

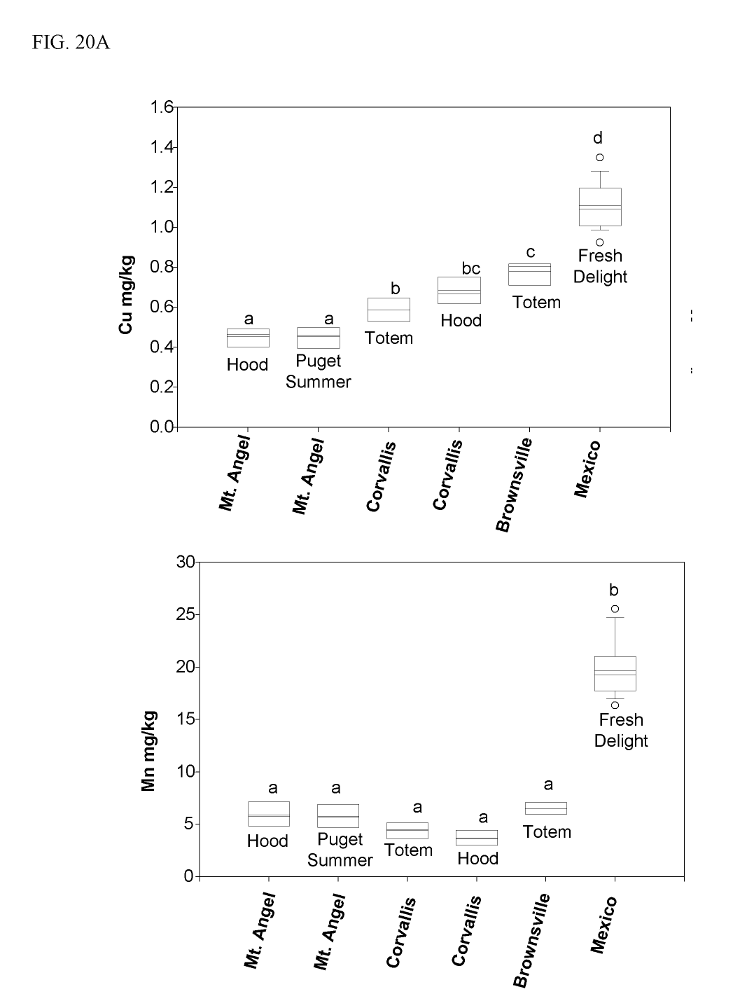

The results of linear discriminate function, quadratic discriminant function, neural network, genetic neural network, and hierarchal tree modeling methods are shown in Table 5. Multiple approaches to evaluate each model included re-substitution, cross-validation, and test-set. The linear discriminant function, the quadratic discriminant function, the neural network, and the genetic neural network models all had a 100% success classification rate for strawberries. The trace elements P, Cu, Zn, and Mg, FIG. 17A, were found to have the most relative importance to the genetic neural network model. The hierarchal tree model also had 100% success rates. Cu concentrations less than 0.87 mg/kg were classified as Mexican (2 terminal nodes), FIG. 18.

TABLE 5
Results of models used to classify the origins of Strawberries, blueberries, and pears

| | | Linear Discriminant | | | Quadratic Discriminant | | |
|---|---|---|---|---|---|---|---|
| | | Function | | | Function | | |
| FRUIT | | Re- | Cross | Test Set | Re- | Cross | |
| (all varieties) | REGION | substitutio | Validation | (n = 10) | substitution | Validation | Test Set |
| Strawberry | Mexico (n = 40) | 100% | 100% | 100% | 100 | 100 | 100% |
| | OR (n = 40) | 100% | 100% | 100% | 100 | 100 | 100% |
| Blueberry | Chile (n = 36) | 100% | 100% | 100% | 100 | 100 | 100% |
| | OR (n = 40) | 100% | 100% | 100% | 100 | 100 | 100% |
| Pear | Argentina (n = 40) | 74% | 75% | 60% | 100 | 100 | 100% |
| | OR (n = 40) | 75% | 70% | 80% | 88% | 85% | 100% |

| | | | | Genetic Neural | | | |
|---|---|---|---|---|---|---|---|
| | | Neural Network | | Network | | Hierarchal Tree | |
| FRUIT | | Re- | Test Set | Re- | Test Set | Re- | Test Set |
| (all varieties) | REGION | substitution | (n = 10) | substitution | (n = 10) | substitution | (n = 10) |
| Strawberry | Mexico (n = 40) | 100% | 100% | 100% | 100 | 100% | 90% |
| | OR (n = 40) | 100% | 100% | 100% | 100 | 100% | 90% |
| Blueberry | Chile (n = 36) | 100% | 100% | 100% | 100 | 100% | 100 |
| | OR (n = 40) | 100% | 100% | 100% | 100 | 100% | 100 |
| Pear | Argentina (n = 40) | 95 | 80% | 100% | 100 | 93% | 90% |
| | OR (n = 40) | 94 | 80% | 100% | 100 | 93% | 90% |

Sample number, linear discriminant function, quadratic discriminant function, neural network, genetic neural network, and hierarchal tree model classification performance analysis results for regional geographical origin prediction of blueberry, strawberry, and pear samples based on total recoverable element concentration profiling.

E. Regional Blueberry Analysis (Oregon vs. Chile)

FIG. 13B depicts variable element concentrations in blueberry samples from Oregon and Chile. Ca, Mg, and Mn were strongly separated (p<<0.0001, 66 d.f.) using a two sample t-test, while Cu, Fe, P, K, Na, and Zn concentrations showed no significant differences between regions (p>0.05). Combinations of Ca, Mg, K, Cu, Na, Fe, or P could be used to visually depict geographic origin group clustering, for example see FIG. 14B. Other elemental combinations were tried; however, these elements gave good visual separation of geographic origin groups. In general, Chilean blueberries had 50% the concentration Mn, and 180% the Ca of Oregon blueberries. One USDA blueberry collection site in Corvallis, Oreg. was excluded (n=8) due to the historical land use at the agricultural experiment station and because no retail commodities are grown for human consumption at this site. Interestingly, blueberries from this experimental site had elevated levels of Cu and Mn (152%, 678% respectively) relative to the average concentrations at the remaining Oregon sites. Although high bush blueberries are not fertilizer intensive, they grow readily in acidic, moist soils. This optimum growing condition renders them susceptible to increased non-nutritive metal uptake. High metal concentrations in blueberries marked this Corvallis site as statistically independent from all others from typical agronomical practices. These data points were removed for further statistical analysis.

Using PCA, 99.9% of the total variability could be explained by the first three principal components (75%, 96%, 99.9% respectively). Strong visual regional clustering was observed for Chilean and Oregon blueberries using PCA (FIG. 15B) and CDA (FIG. 16B).

F. Blueberry Modeling

The results of linear discriminant function, quadratic discriminant function, neural network, genetic neural network, and hierarchal tree modeling methods are shown in Table 5. The linear discriminant function, the quadratic discriminant function, the neural network, and the genetic neural network models all had a 100% success classification rate for blueberries. The trace elements Cu, Mg, and Zn, FIG. 17B, were found to have the most relative importance to the genetic neural network model. The hierarchal tree model had a 100% success rates. Mn concentrations <6.65 mg/kg were classified as Chilean (2 terminal nodes), FIG. 18.

G. Regional Pear Analysis (Oregon vs. Argentina)

Element concentration variation in Oregon and Argentine pears can be observed in FIG. 13C. Two sample t-tests suggests that Cu concentration showed significant separation (p<0.0001, 78 d.f.) as did Ca (p<0.01), while all other element concentrations were not significantly different between Oregon and Argentine pears (p >0.05). Combinations of trace elements could not be found that provided good visually depicted geographic origin group clustering. PCA results showed that the first three components explained 99.9% of the variability (93%, 98%, 99.9% respectively); however, no visual clustering of Oregon and Argentine pears was observed, FIG. 15C. Canonical discriminant analysis frequency chart for Oregon and Argentina pears also shows a great deal of overlap, FIG. 16C.

H. Pear Modeling

The results of linear discriminate function, quadratic discriminant function, neural network, genetic neural network, and hierarchal tree modeling methods are shown in Table 5. Multiple approaches to evaluate each model included re-substitution, cross-validation and test-set as shown in Table 5. Overall, the linear discriminant function model did not perform very well on the pear data set; this modeling analysis had only a 60-80% success rate. The other modeling methods were more successful. The quadratic discriminant function had an 85 to 100% success rate, and the neural network had an 80-95% success rate. The best model for the pear data set was the genetic neural network models, which had a 100% success rate. Genetic algorithms seek to solve optimization problems using the methods of evolution, explicitly survival of the fittest. In a typical optimization problem, there are a number of variables, which control the process, and a formula or algorithm, which combines the variables to fully model the process. The problem is then to find the values of the variables that optimize the model in some way. Other traditional methods tend to break down when the problem is not so “well behaved,” but genetic algorithms are designed to perform on data that is not so “well behaved” which may account for its success with pears. The micro trace elements Cu, Mn, and V, FIG. 17C, were found to have the most relative importance to the genetic neural network model. The hierarchal tree model used for regional classification prediction of Oregon and Argentine pears is shown in FIG. 18 and is significant more complex than for the other fruit tested. The tree model requires 8-terminal nodes to meet the classification criteria and then has a classification success rate of 93%.

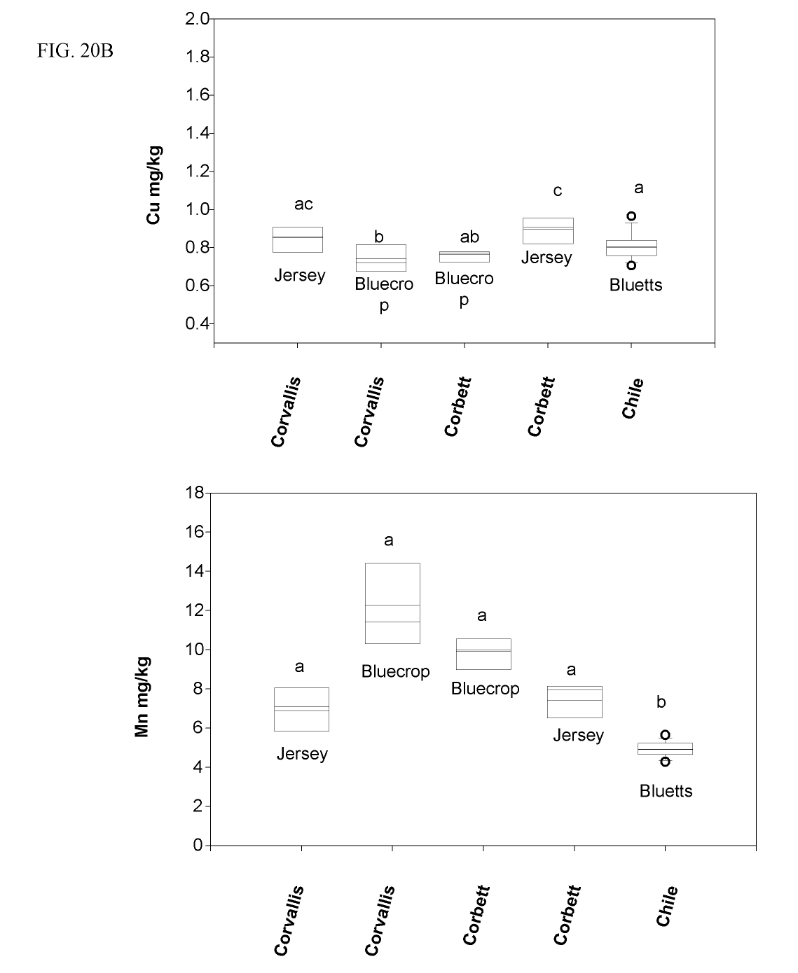

Investigation of bulk stable isotope ratios in pear samples was used to address the lack of initial modeling success between Oregon and Argentine samples. Bulk stable isotope ratios, δ¹³C/δ¹⁵N, depicts visual separation between Oregon and Argentine pears, shown in FIG. 19. Oregon pears had significantly less enrichment of lighter ¹²C than Argentine pears (p<0.0001, 42 d.f.). No significant differences in δ¹⁵N were observed between Oregon and Argentine pears (p>0.05). Addition of the bulk stable isotope ratio data to the models would most likely increase the modeling success rate for pears.

I. Variety and Sub-Regional Analysis

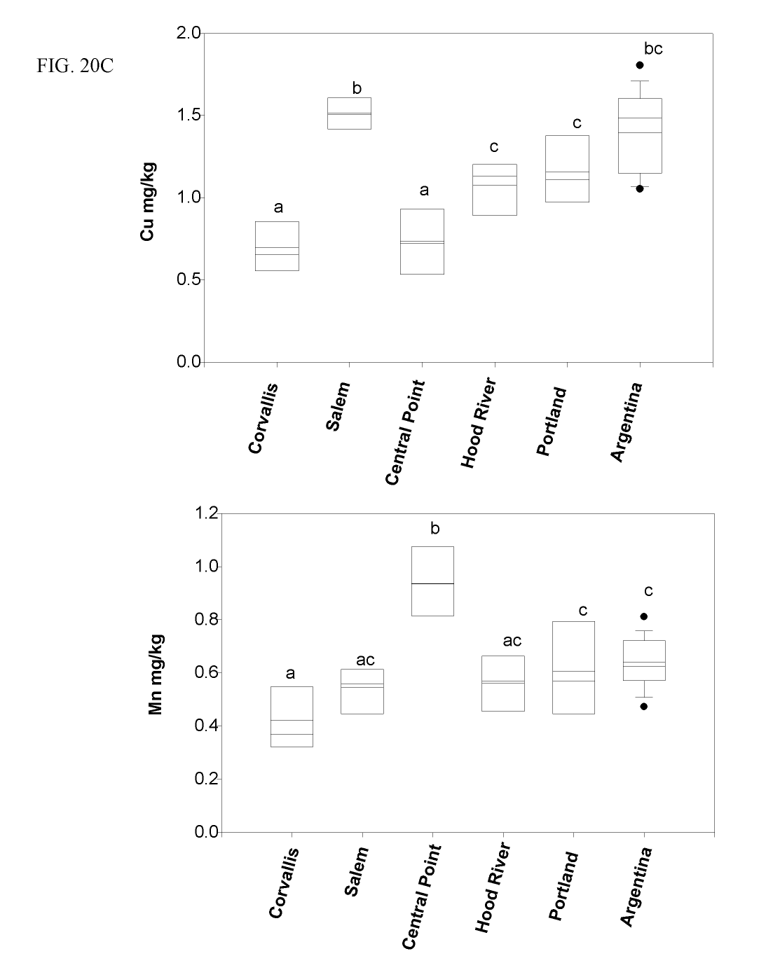

One caveat of using profiles of elemental concentrations based on country-to-country data is the possibility of misclassification due to variety. It is difficult to get good variety data, because typically each variety is grown in a different location, so there are inherent geographic differences leading to large confounding variables. Two varieties of strawberries and two varieties of blueberries were collected, from adjacent plants (same soil, same environment, same agronomy practices) therefore providing an excellent opportunity to evaluate the variety effect without many of the typical confounding variables. Effects of variety on geographic origin analysis of strawberries and blueberries were previously unknown. FIG. 20A-20C shows some of the element variety and sub-regional differences, suggesting differing variety element uptake for both strawberries and blueberries.

J. Oregon Strawberry Variety Effects

Although there are large differences in Cu and Mn concentrations between Mexican and Oregon strawberry; there are also some Cu and Mn variety differences between Oregon strawberries, FIG. 20A (multiple comparisons ANOVA). Significant Na concentration differences were seen between Totem and Hood cultivars from the S. Corvallis field location (p<0.01). Fe concentrations were significantly different between Hood and Puget summer cultivars at the Mt. Angel field site (p<0.01). Although there are variety differences within Oregon strawberry grown in the same field, these differences are relatively small compared to the overall elemental profile differences with Mexican strawberries, and most importantly within the framework of this study do not appear to adversely affect modeling success, Table 6.

TABLE 6
Results of models used to classify the variety of Strawberries and blueberries.

| | | Linear | Quadratic | | Genetic | |
|---|---|---|---|---|---|---|
| | | Discriminant | Discriminant | Neural | Neural | Hierarchal |
| FRUIT | | Functio | Functio | Network | Network | Tree |
| (all varieties) | VARIETY | Average Test Set | | | | |
| Strawberry | hood | 100% | 100% | 100% | 100% | 88% |
| | tote | 100% | 100% | 100% | 100% | 100% |
| Blueberry | bluecrop | 100% | 100% | 100% | 100% | 100% |
| | jersey | 100% | 100% | 100% | 100% | 63% |

*Varieties selected for Test set modeling were those from field sites where two varieties were sites where only a single variety was available (Mt. Angel: Puget summer, Hood; Brownsville: not modeled

Linear discriminant function, quadratic discriminant function, neural network, genetic neural network, and hierarchal tree model classification performance analysis results for varietal effects for geographical origin prediction of blueberry, strawberry, and pear samples based on total recoverable element concentration profiling.

The affects of variety on all of the models was tested. At field sites with two varieties one variety was removed from the model training set. The training set then contained some strawberries from the geographic site (representing environmental conditions, soil, agronomical practices, etc.) but would not contain the second variety, in this way isolating the variety effect. The test set would then be composed of a single variety, as always, withheld from the training set. The linear discriminant function, quadratic discriminant function, neural network, and genetic neural network models all had 100% success rates, Table 6. The hierarchal tree model had 88 to 100% success rates.

K. Oregon Strawberry Sub-regional Effects

The strawberry cultivar Totem had significantly higher mean Zn concentration at the Brownsville site compared to the Corvallis site only 22 miles away (p<0.01). Significant mean concentration differences among the Hood cultivar between Corvallis and Mt. Angel field locations were also seen for Cu, K, and Zn (p<0.0001). These sub-regional differences are not surprising, considering the diversity of Oregon soils. However, like the variety data, within this strawberry dataset, sub-regional differences are relatively small and do not adversely affect geographic origin modeling combined with profile elemental concentration; hierarchal tree model test set success rates were greater than 88% (Brownsville 88%, Corvallis 94%, and Mt. Angel 100%).

L. Oregon Blueberry Variety Effects

Significant difference in element concentrations among blueberries, Jersey variety and Bluecrop cultivar, suggests there are discernable differences between varieties/cultivars of blueberry picked from the same field location, as is the case with Cu shown in FIG. 20B. Mean element concentrations of Cu and Zn were significantly different between Jersey and Bluecrop blueberries at the Corvallis field location (p<0.0001). Jersey and Bluecrop blueberries also showed significant differences between mean Ca, Cu, and Mg picked from the Corbett field site (p<0.005). Variety test sets were created as described above for strawberry. The linear discriminant function, quadratic discriminant function, neural network, and genetic neural network models all had 100% success rates, Table 6. The hierarchal tree model had 63 to 100% success rates. This suggests that within this blueberry data set that variety/cultivar differences do not adversely affect most geographic origin modeling using profile elemental concentrations. The hierarchal tree model however, did not perform as well overall within the framework of this study, suggesting blueberry variety/cultivar may adversely affect some models.

M. Oregon Blueberry Sub-regional Effects

The Bluecrop cultivar showed significant differences between the Corvallis and Corbett field sites for mean Ca, Mg, Mn, K, and Zn concentrations (p<0.05). The Jersey variety showed a significant difference between the Corvallis and Corbett sites only for mean Ca and Mg concentrations (p<0.04). Similar success rates were achieved on sub-regional test sets (>80%). Models could also be created with a high degree of success based on sub-regional geographic origins. Hierarchal tree model test set success rates were greater than 82% (Corvallis 82%, Corbett 88%).

N. Oregon Pear Sub-regional Analysis

Differences in metal concentrations among Bartlett pear samples from Oregon sub-regions can be seen in FIG. 20C and are small relative to strawberries and blueberries. Tree fruits undergo a more significant element translocation distance and reproductive sinks are directly related to the age of the tree and climate of the growing site. In spite of these results, significant differences were observed between sites. The most dramatic differences were found with Cu concentrations at Salem (p<0.001) and with Mn concentrations at Central Point (p<0.001); with respect to the Portland site (the next site closest in concentration for both metals). Hierarchal tree model test set success rates were greater than 50% (Central Pt. 63%, Corvallis 75%, Salem 50%, Portland 50%, and Hood River 100%). Only one variety of pears was included in the study, so variety analysis was not performed.

O. Pear Bulk Stable Isotope Ratios

Because the modeling of element profiles for the pears was less successful, bulk stable isotope ratios as a means to gain further discriminating chemical data was investigated. Isotopic analysis of Oregon sub-locations showed significant separation among δ¹⁵N ratios, ranging from −2 to +4δ¹⁵N. Most Oregon sub-locations δ¹⁵N ratios were significantly different from one another. Central Point showed strong significant differences from all other sub-locations (p<0.01), while Hood River was significantly different from Portland and Salem (p<0.05). Portland and Salem were not statistically different from one another (p>0.05). Positive δ¹⁵N ratios indicate a selective enrichment of heavy ¹⁵N compared to ¹⁴N. Central Point Bartlett pear samples accumulated the heavier ¹⁵N isotope compared to ¹⁴N followed by Salem, Portland, and Hood River respectively. This could be in part, due to the latitudinal differences of the field sites, δ¹⁵N has been associated with latitude.

Another caveat of the pear dataset was potentially revealed when sub-regional geographic origin CDA plots were generated. Oregon pears show visual clustering differences from Argentine pears, with one notable exception, the pears labeled as from ‘Portland’. These were the only samples from Oregon not hand collected. One possible explanation for this overlap could be that these pears were mislabeled as being grown in Oregon. As with all authenticity studies, authenticating samples is critical to the study, as well as developing a data base that contains all the potential variation in the population to be studied. The genetic neural network model performed the best of all modeling methods. Interestingly, some elements from the genetic neural network model were consistently found to be important to the model input, specifically Cu, Mn, Mg, and Zn. It may be possible to create further simplification of the method by analyzing and modeling only these elements and as needed adding bulk stable isotopes. Creating a fingerprint or unique chemical signature using trace element and stable isotope ratio chemical profiling can serve as a cost effective approach toward determining the geographic growing region of a food commodity. The identification of distinct chemical-signature effects on geographic origin from sub-location and variety/cultivar of fresh fruits has not previously been described. The ease and efficiency of trace metal analysis, makes it an optimal choice for geographic regional and sub-regional determination of blueberries, strawberries, and pears. Within the framework of this study, it appears that the geographic origin of strawberries, blueberries, and pears may be determined by their chemical profile. Statistical analyses revealed groupings between the two major geographic regions for each commodity studied. The progression of this type of profiling study includes the addition of other geographic regions, seasonal variation (including agronomical changes) and additional varieties from all locations.

### EXAMPLE 4

### This Example Describes Stable Isotope Ratio Analysis of Salmon

Samples of salmon from know origins were collected. Farmed salmon of known origin samples were collected from outlets throughout Oregon and the Pacific Northwest. Samples of “wild” salmon were obtained directly from the local outlets as necessary though out the “wild” salmon fishing season. Samples from non-local outlets were purchased directly by associates and shipped to the laboratory for analysis. Chain-of-custody is maintained for all samples. Approximately 100 known samples were collected, comprising roughly ⅓ each of wild Pacific salmon, Pacific farmed salmon, and Atlantic farmed salmon.

Samples were stored at <−10° C. until analysis. Sample processing may include, but is not limited to, homogenization under liquid nitrogen, drying and homogenization, or freeze drying and homogenization. Subsamples of the processed samples were digested and refluxed with concentrated acid under heat not to exceed 130° F. for approximately 7 hours. The digestate was diluted to 10 mL final volume, vortexed and filtered. This final solution was stored at room temperature until further processing for metal analysis. Metal quantitation on the diluted digestate can be accomplished using Inductively Coupled Plasma (ICP) Optical Emission Spectrometer (OES) or mass spectrometry (MS).

The samples are normally dried (or freeze dried) and ground and can be maintained at room temperature before analysis. Analytical conditions, e.g. high or low dilution, require knowledge of sample matrix and relative C and N quantity. Standard precision is approx. ±0.02‰, but are checked on a routine basis. For stable isotope analysis, the fish tissue was weighed into a capsule and analyzed by mass spectrometric analysis (MS). The stable isotope ratios (¹³C/¹²C, ¹⁵N/¹⁴N, ¹⁸O/¹⁶O) were measured in wild salmon, farmed salmon from the West coast of the United States, and farmed salmon from the East coast of the United States. Stable isotope ratios (¹³C/¹²C, ¹⁵N/¹⁴N, ¹⁸O/¹⁶O) were tested, calibrated, and differences between the farmed and wild salmon were determined. Stable isotope ratios (¹³C/¹²C, ¹⁵N/¹⁴N, ¹⁸O/¹⁶O) are tested, calibrated, and differences between Pacific framed versus Atlantic farmed salmon were determined. Nitrogen (δ¹⁵N‰), oxygen (δ¹⁸O‰) and carbon (δ¹³C‰) stable isotopes and bulk C/N ratios were measured on a stable isotope mass spectrometer (MS) (Finnigan MAT-251, ThermoFinnigan, Waltham, Mass.). Isotopic data use the standard isotopic delta notation (δ), in per mil (‰), relative to the Pee Dee Belemnite (PDB) scale for carbon isotopes, relative to air (¹⁵N) for nitrogen, and standard mean ocean water for ¹⁸O. By convention, the following equation for delta is used for carbon (and analogous equations for nitrogen and oxygen):

Delta(δ)¹³C‰=[(¹³C/¹²Csample)−(¹³C/¹²Cstd)/(¹³C/¹²Cstd)]×1000

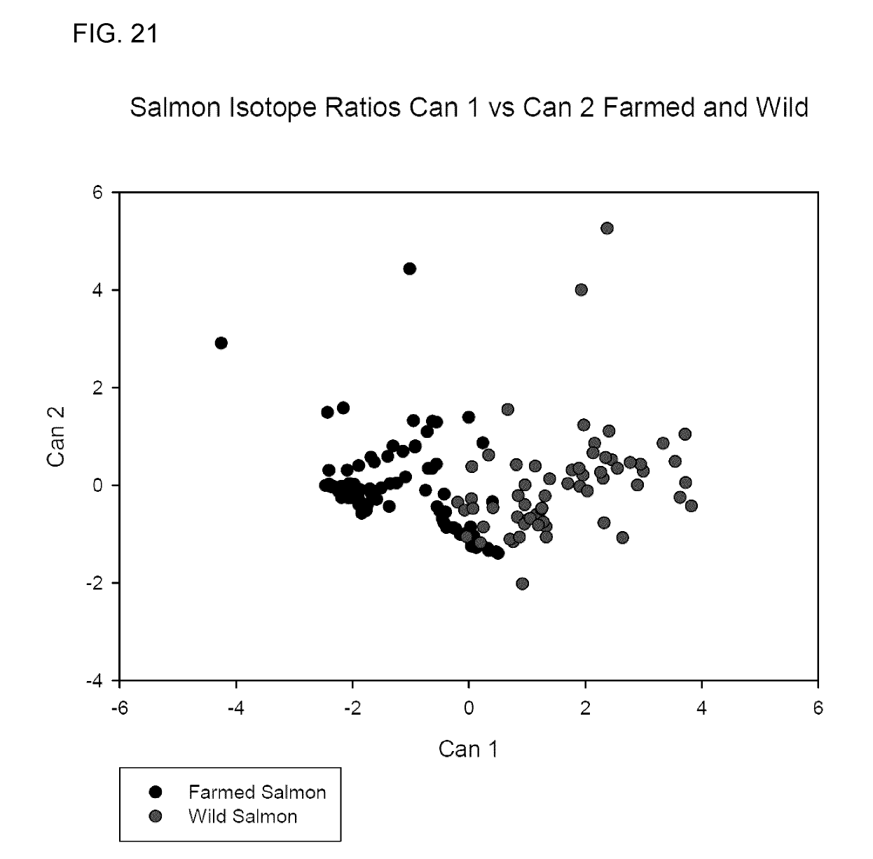

As shown in FIG. 21A when the obtained mass-spec data is subjected to canonical variate analysis, a plot of the first two canonical variables reveals that the farmed salmon and the “wild” salmon segment into two distinct regions of the plot. This result demonstrates that isotope ratios can be used to discriminate between “wild” and farm raised salmon. Stable isotopes ratios from salmon are used for developing databases correlating isotope ratios to origin.

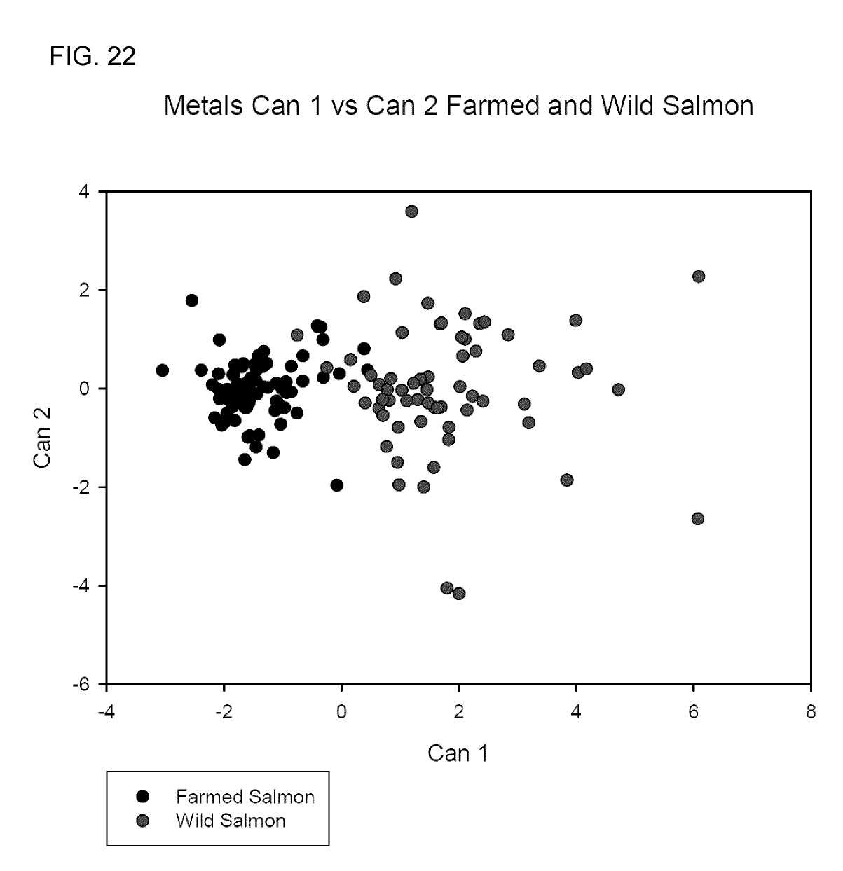

The trace metals in samples of “wild” and farm raised salmon were also examined for a correlation with origin. Trace metals were analyzed as previously described for pistachios in Example 1. As sown in FIG. 22, when the obtained elemental analysis data is subjected to canonical variate analysis, a plot of the first two canonical variables reveals that the farmed salmon and the “wild” salmon segment into two distinct regions of the plot. This result demonstrates that obtained elemental analysis can be used to discriminate between “wild” and farm raised salmon. Elemental profiles from salmon are used for developing databases correlating isotope ratios to origin.

A hierarchical tree of decision rules is constructed using the isotope ratios and/or elemental analysis for salmon correlated to origin can be constructed.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method for determining the origin of a food product, comprising:
providing a food product of unknown origin;
determining at least one stable isotope ratio of the food product of unknown origin;
optionally determining the concentration of at least one trace element of the food product of unknown origin; and
predicting origin of the food product of unknown origin by comparing the at least one stable isotope ratio of the food product and optionally the concentration of at least one trace element of the food product to a standard.

2. The method according to claim 1, where the standard comprises at least one stable isotope ratio of at least one food product of known origin, and optionally the concentration of at least one trace element of the at least one food product of known origin, correlated to the origin of the at least one food product of known origin.

3. The method according to claim 2, where the standard is determined by a method comprising:
providing at least one food product of known origin having at least one stable isotope ratio;
determining at least one stable isotope ratio of the at least one food product of known origin;
optionally determining the concentration of at least one trace element of the at least one food product of known origin; and
correlating at least one stable isotope ratio of the at least one food product of known origin, and optionally the concentration of at least one trace element of the at least one food product of known origin, to the origin of the at least one food product of known origin.

4. The method according to claim 1, where the origin includes geographic origin, seasonal origin, environmental origin, production method, or combinations thereof.

5. The method according to claim 3, where correlating the at least one stable isotope ratio of the at least one food product to the origin of the at least one food product and the optional concentration of at least one trace element of the at least one food product comprises using principal component analysis (PCA), canonical discriminant analysis (CDA), linear discriminant function analysis, quadratic discriminant function analysis, neural network modeling, genetic neural network modeling, or combinations thereof.

6. The method according to claim 1, where determining stable isotope ratios of elements comprises determining ¹³C/¹²c, ¹⁵N/¹⁴N, ¹⁸O/¹⁶O, ²H/¹H ratios, or combinations of such ratios.

7. The method according to claim 1, where the isotope ratios include ¹³C/¹²C and ¹⁵N/¹⁴N.

8. The method according to claim 1, where determining stable isotope ratios of elements comprises determining carbon and nitrogen isotopes where δ¹³C is measured as CO₂ and δ¹⁵N is measured as N₂.

9. The method according to claim 1, where the isotope ratios are determined by comparing CO₂ and N₂ isotope ratios.

10. The method according to claim 1, where determining stable isotope ratios includes determining δ¹⁵N values.

11. The method according to claim 1 comprising determining bulk C/N ratios.

12. The method according to claim 1 comprising correlating C/N ratio versus δ¹⁵N‰ to origin.

13. The method according to claim 1 comprising determining both trace element concentration of at least one trace element and a stable isotope ratio of at least two isotopes.

14. The method according to claim 1 comprising determining concentrations of plural trace elements.

15. The method according to claim 1, where determining concentration of at least one trace element of the at least one food product comprises determining trace element concentrations of Ca, Cu, Fe, K, Mg, Mn, Na, P, Sr, V, Zn, or combinations thereof.

16. The method according to claim 1, comprising correlating origin based on measured profile of trace element concentrations found in the food product.

17. The method according to claim 1, comprising correlating seasonal origin by comparing element distributions by season for a given region.

18. The method according to claim 1, comprising applying principal component analysis to normalize trace element data.

19. The method according to claim 1, including determining concentration of at least one trace element, and where canonical discriminant analysis (CDA) is used to obtain group clustering.

20. The method according to claim 1, where the food product is a commodity.

21. The method according to claim 1, where the food product is a fruit, vegetable, nut, grain or cereal.

22. The method according to claim 1, where the food product is an animal product.

23. The method according to claim 22, where the animal product is from fish, fowl, swine or ruminant.

24. A method for correlating a food product with the origin of the food product comprising:
providing at least one food product of known origin;
determining at least one stable isotope ratio of the at least one food product;
optionally determining the concentration of at least one trace element of the at least one food product; and
correlating at least one stable isotope ratio of the at least one food product and optionally the concentration of at least one trace element of the at least one food product to the origin of the at least one food product.

25. The method according to claim 24, where the origin includes geographic origin, seasonal origin, environmental origin, or combinations thereof.

26. The method according to claim 24 where correlating the at least one stable isotope ratio of the at least one food product to the origin of the at least one food product and the optional concentration of at least one trace element of the at least one food product comprises using principal component analysis (PCA), canonical discriminant analysis (CDA), linear discriminant function analysis, neural network modeling, genetic neural network modeling, or combinations thereof.

27. The method according to claim 24, where determining stable isotope ratios of elements comprises determining ¹³C/¹²C, ¹⁵N/⁴N, ¹⁸O/¹⁶O, ²H/¹H ratios, or combinations of such ratios, of compounds that are formed in the organisms.

28. The method according to claim 24, where the isotope ratios include ¹³C/¹²C and ¹⁵N/¹⁴N.

29. The method according to claim 24, where determining stable isotope ratios of elements comprises determining carbon and nitrogen isotopes where δ¹³C is measured as CO₂ and δ¹⁵N is measured as N₂.

30. The method according to claim 24, where the isotope ratios are determined by comparing CO₂ and N₂ isotope ratios.

31. The method according to claim 24, where determining stable isotope ratios includes determining δ¹⁵N‰ values.

32. The method according to claim 24, comprising determining bulk C/N ratios.

33. The method according to claim 24, comprising correlating C/N ratio versus δ¹⁵N to origin.

34. The method according to claim 24, comprising determining both trace element concentration of at least one trace element and a stable isotope ratio of at least two isotopes.

35. The method according to claim 24, comprising determining concentrations of plural trace elements.

36. The method according to claim 24, where determining concentration of at least one trace element of the at least one food product comprising determining trace element concentrations of Ca, Cu, Fe, K, Mg, Mn, Na, P, Sr, V, Zn, or combinations thereof.

37. The method according to claim 24, comprising correlating origin based on measured profile of trace element concentrations found in the food products.

38. The method according to claim 24, comprising correlating seasonal origin by comparing element distributions by season for a given region.

39. The method according to claim 24, comprising applying principal component analysis to normalized trace element data.

40. The method according to claim 24, including determining concentration of at least one trace element, and where canonical discriminant analysis (CDA) is used to obtain group clustering.

41. The method according to claim 40, where CDA analysis was applied to element concentrations for Sr, Cu, Na, Ca, Fe, and Cu.

42. The method according to claim 24, where the correlation of at least one stable isotope ratio of the at least one food product and optionally the concentration of at least one trace element of the at least one food product to the origin of the at least one food product is stored on computer readable media.

43. The method of claim 24, further comprising generating an algorithm based on the correlation of the at least one stable isotope ratio of the at least one food product and optionally the concentration of at least one trace element of the at least one food product to the origin of the at least one food product.

44. The method of claim 24, further comprising assembling the correlation of at least one stable isotope ratio of the at least one food product and optionally the concentration of at least one trace element of the at least one food product to the origin of the at least one food product into a database.
